(19) United States
(12) Patent Application Publication
(10) Pub. No.: US 2019/0023760 A1
(43) Pub. Date: Jan. 24, 2019

(54) METHOD FOR PREPARING INTERLEUKIN-2 OR INTERLEUKIN-2 ANALOGUES

(71) Applicant: ETH ZURICH, Zuerich (CH)

(72) Inventors: Jeffrey BODE, Zuerich (CH); Claudia MURAR, Zuerich (CH); Mamiko NINOMIYA, Zuerich (CH)

(73) Assignee: ETH ZURICH, Zuerich (CH)

(21) Appl. No.: 15/879,330

(22) Filed: Jan. 24, 2018

(30) Foreign Application Priority Data
Jul. 24, 2017 (EP) 17182851.0

(51) Int. Cl. C07K 14/55; C07K 14/00; C07K 1/00

(52) U.S. Cl. CPC C07K 14/55; C07K 1/003; C07K 14/001

(57) ABSTRACT

A method for preparing interleukin-2 or an interleukin-2 analogue formed by at least three building blocks including the steps of: a) Synthesizing the at least three building blocks, whereby the C-terminal amino acid of each building block is linked to an a-keto group and the N-terminal amino acid of each building block is linked to a cyclic hydroxylamine, b) Coupling the at least three building blocks by KAHA ligation resulting in a depsipeptide, c) Rearrangement of the depsipeptide to obtain interleukin-2 or an interleukin-2 analogue.

The present invention relates to a method for preparing interleukin-2 or interleukin-2 analogues.

The protein interleukin-2 is a cytokine that was originally described as permitting the activation and proliferation of T lymphocytes. It has been clinically used for the stimulation of effector immune response in certain cancers and infectious diseases, Interleukin-2 was approved under the tradename Proleukin® in 1998 for the treatment of metastatic renal cell carcinoma. Human Interleukin-2 contains 133 amino acids including three cysteines (SEQ. ID. NO. 1). Two of said cysteines form an intramolecular disulfide bridge and the third one has a free sulfhydryl group and is not involved in the biological activity of the protein.

Therapeutic interleukin-2 is typically prepared by recombinant techniques from Escherichia coli. However, the expression and purification is known to be problematic due to the formation of insoluble, improperly folded aggregates, and therefore yield is poor.

The chemical synthesis is an important method for preparing proteins of biological interest. Native chemical ligation (NCL) developed by Kent (U.S. Pat. No. 6,184,344) allows the synthesis of proteins containing more than 100 amino acids. However, said method is not always suitable for the preparation of proteins having a hydrophobic region. Due to the hydrophobic terminal region of interleukin-2, the synthesis of said protein not possible by native chemical ligation.

Another method for preparing peptides is the so-called α-ketoacid-hydroxylamine (KAHA) ligation (Pattabiraman, R.; Bode, J. W.: Rethinking amide bond synthesis. Nature 2011, 480, 471-479.). The KAHA ligation is a chemoselective way to couple two unprotected peptides, one bearing a C-terminal u-ketoacid functional group and the other an N-terminal hydroxylamine forming an amide bond at the ligation site. For example, Harmand et al disclose in Nature Protocols 2016, 11, 1130-1147 the total chemical synthesis of the mature betatrophin.

Although interleukin-2 is a globular glycoprotein, its C-terminal region, in particular amino acids at positions 99 to 133, is extremely insoluble when synthesized by solid phase peptide synthesis (SPPS) as the peptide segment has a strong tendency to aggregate. Asahina et al (Angew. Chem. Int. Ed. 2015, 54, 8226-8230) disclose a highly complex chemical synthesis of human interleukin-2 involving many steps and effort. Said method is not suitable for a technical scale-up.

Interleukin-2 suffers from poor stability since it is susceptible to degradation in the presence of water and oxygen and reducing reagents such as reduced glutathione. According to the prior art, interleukin-2 may undergo chemical degradation and physical instability in solution. In order to avoid such degradation, lyophilized formulations and formulations comprising antioxidants and 1:Preservatives were developed. For example, WO 2017/068031 discloses such a formulation.

The problem of the present invention is to provide a method for preparing interleukin-2 or an interleukin-2 analogue having an increased stability,

The problem is solved by the method according to claim 1. Further preferred embodiments are subject of the dependent claims.

### BRIEF DESCRIPTION OF THE DRAWINGS

FIG. 1a shows the interleukin-2 sequence (SEQ. 10. No.1) assembled by four building blocks whereby building is block 1 is formed by amino acids 1 to 40 (SEQ. 10. No. 31),building block 2 is formed by amino acids 41 to 70 (SEQ. 10. No. 32 or SEQ. 10. No. 35),building block 3 is formed by amino acids 71 to 103 (SEQ. 10. No. 33) and building block 4 is formed by amino acids 104 to 133 (SEQ. 10, No. 34, SEQ. 10 No, 30, SEQ. 1.0 No. 36 or SEQ. 10 No. 37);

FIG. 1b shows the synthesis of interleukin-2 by assembling four building blocks via KAHA ligations followed by cysteine deprotection;

FIG. 2 shows an interleukin-2 analogue containing PEN residues;

FIG. 3 shows an interleukin-2 analogue containing a methylene thioacetal bridge instead of the Cys58-Cys 105 disulfide inter bridge of interleukin-2.

The method for preparing interleukin-2 or an interleukin-2 analogue formed by at least three building blocks involves the following steps
a. synthesizing the at least three building blocks, whereby the C-terminal amino acid of each building block is linked to an a-keto group and the N-terminal amino acid of each building block is linked to a cyclic hydroxylamine,
b. coupling the at least three building blocks by KAHA ligation resulting in a depsipeptide,
c. rearrangement of the depsipeptide and folding to obtain interleukin-2 or an interleukin-2 analogue.

The method according to the present invention results in the production of interleukin-2 or interleukin-2 analogues by KAHA ligation in very high yield, preferably with an overall yield for the KAHA ligations of more than 259, most preferably more than 30%. In fact, with the method is according to the present invention one Man prepare any kind of analogues with modified amino acids.

Preferably, the cyclic hydroxylamine linked to the N-terminal amino acid of each building block is 5-oxaproline or oxazetidine, most preferably 5-oxaproline because it is better accessible.

Preferably, interleukin-2 or the interleukin-2 analogue is divided into 3 to 8, more preferably into 4 building blocks. If, for example, three building blocks are present, two KAHA ligations have to take place. Preferably, the building blocks have roughly equal size, that is they consist of about the same number of amino acids. Preferably, each building block has 25 to 45 amino acids.

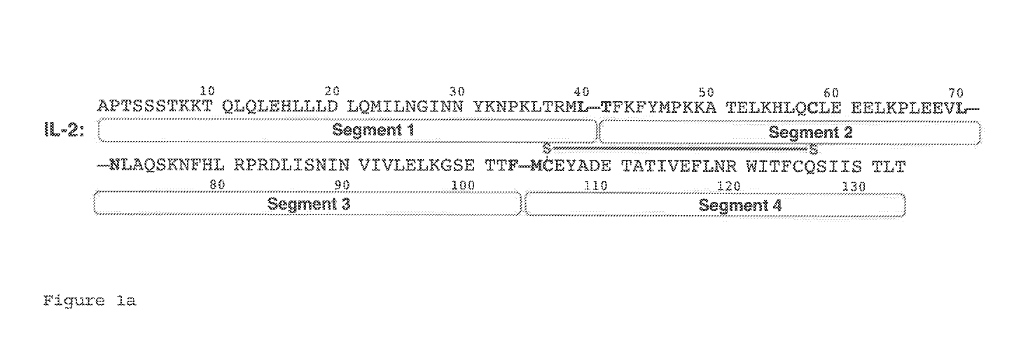

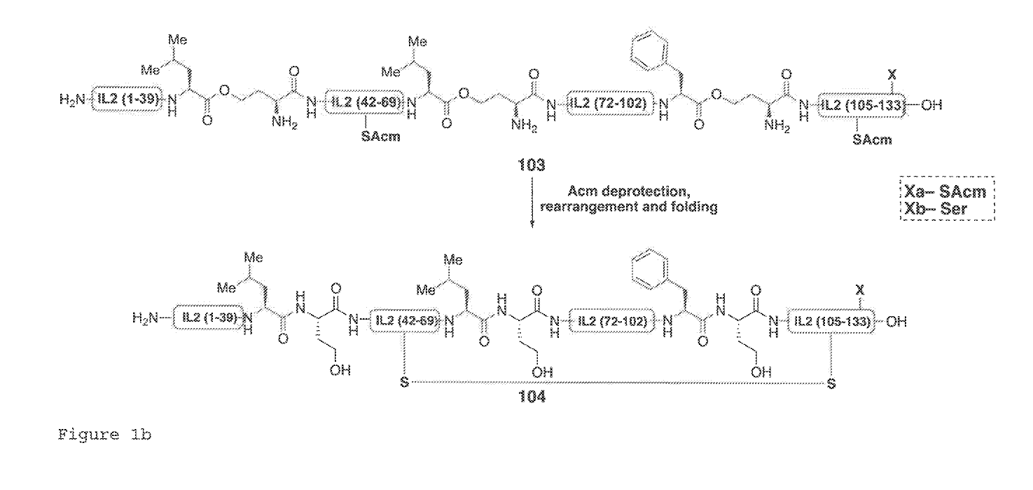

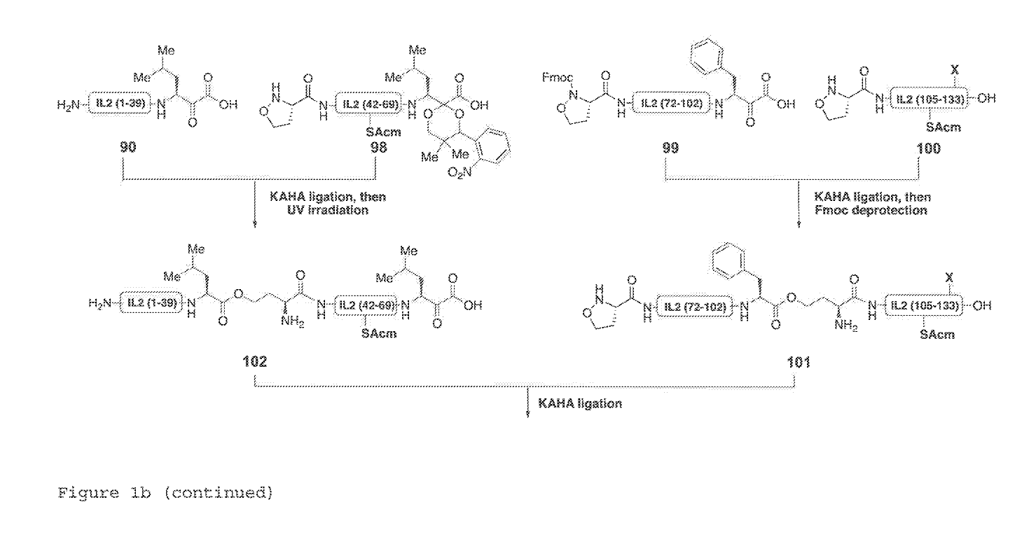

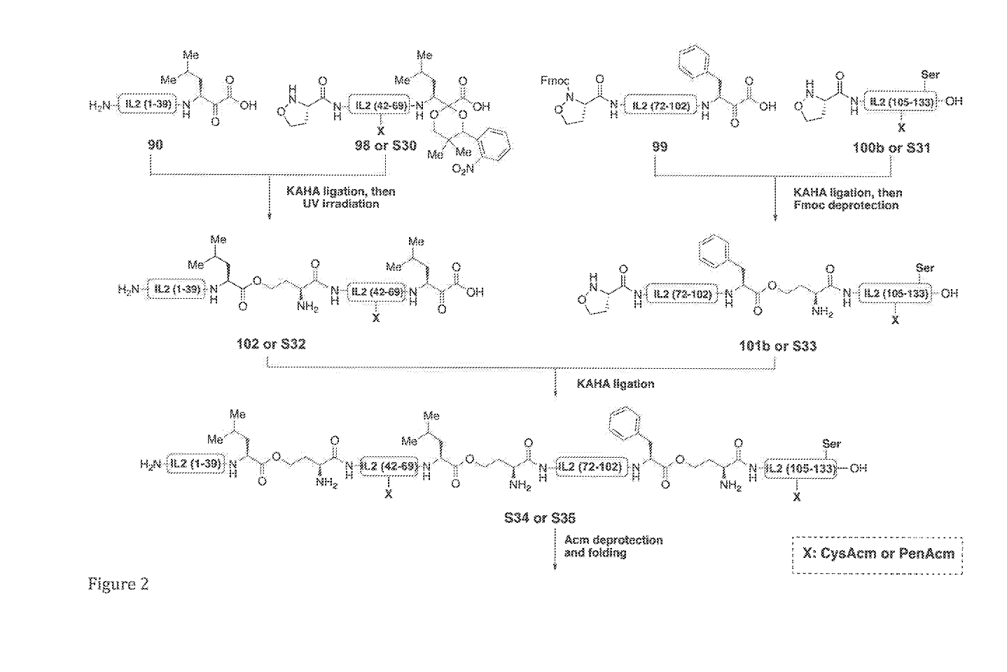

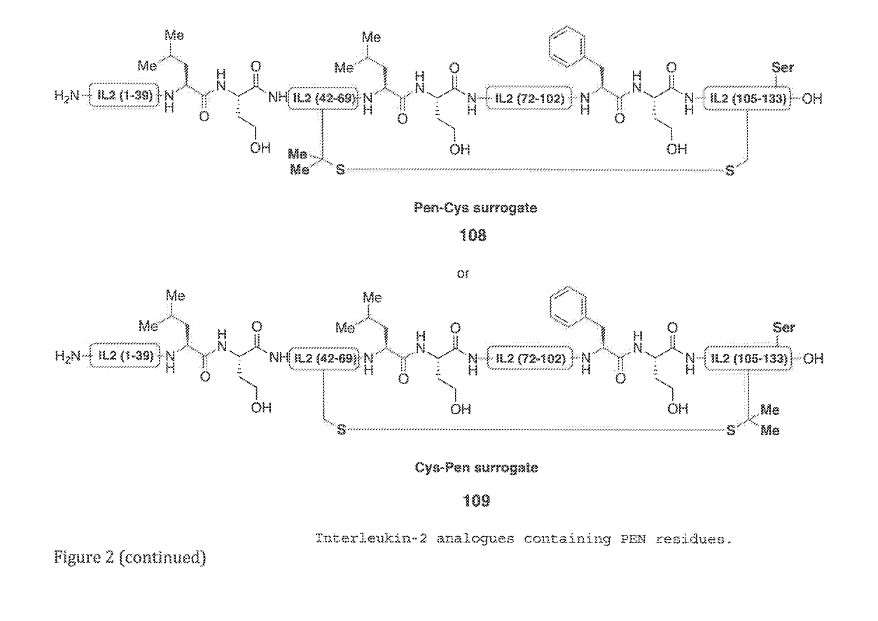

Very good results could be obtained by forming the interleukin-2 or the interleukin-2 analogues with 4 building blocks as shown in FIG. 1a, and whereby
building block 1 is formed by amino acids 1 to 40 (SEQ. ID. No. 31),
building block 2 is formed by amino acids 41 to 70 (SEQ. ID. No. 32 or SEQ. ID. No. 35),
building block 3 is formed by amino acids 71 to 103 (SEQ. ID. No. 33) and
building block 4 is formed by amino acids 104 to 133 (SEQ. ID. No. 34, SEQ. ID No. 30, SEQ. ID No. 36 or SEQ. ID No. 37),

whereby the amino acid numbers refer to the interleukin-2 sequence (SEQ. ID. No. 1).

Preferably, the C-terminus of the at least three, more preferably four building blocks to be coupled with the N-terminus of the next building block is selected from the group consisting of leucine, phenylalanine, valine, tyrosine, arginine, glutamine, alanine, norleucine and isoleucine, preferably of leucine, phenylalanine, valine, tyrosine and arginine, and most preferably of leucine, phenylalanine and valine. The ligation sites include preferably leucine, phenylalanine, valine, tyrosine and arginine c-keto groups as the most effective ligation partners for 5-oxaproline. Of course the last building block of the C-terminus of the protein is typically threonine (amino acid 133).

Preferably all cysteines at positions 58, 105 and 125 are replaced by cysteine S-acetamidomethyl (CysAcm) in order to increase the stability of the building blocks The acetamidomethyl protecting group can be removed before forming the disulfide-bridge, for example, by treating a diluted solution of the interleukin-2 in a 1:1 mixture of water and acetic acid with 1% silver acetate (AgOAc) for 2 hours at 50° C.

The synthesis could be further improved by introducing isoacyldipeptide or depsipeptides for Ile129-Ser130 (Boc-Ser-Ile(Fmoc)-014), in order to increase the solubility of the protein, and thus facilitating the elongation on the resin and the synthetic yield. The native sequence is regenerated at the very end during the rearrangement by exposing the protein in a basic buffer.

Alternatively or in addition, pseudoproline dipeptide (Fmoc-Ala-Thr(Psi(Me,Me)pro)-OH) may be introduced for Ala112-Thr113 in order to decrease the agregation of the protein, and thus facilitating the elongation on the resin and the synthetic yield. The native sequence is regenerated by deprotection during the cleavage conditions from the resin using TFA cocktail (TFA:TIPS:water).

Another object of the present invention is to provide new interleukin-2 analogues. Such interleukin-2 analogues can be obtained by replacing at least one or all methionine amino acids at positions 23, 39 and 46 of the interleukin-2 sequence (SEQ. ID. NO. 1) by norleucine (Nle) in order to avoid oxidation while handling, storage and refolding as said methionine residues are not essential for bioactivity in the case interleukin. These modifications results in interleukin-2 analogues SEQ. ID. NO. 7, SEQ. ID. NO. 8, SEQ. ID. NO. 9, SEQ. ID. NO. 10, SEQ. ID. NO. 11, SEQ. ID. NO. 12, and SEQ. ID. NO. 13.

By replacing one of the disulfide bond forming cysteines at positions 58 and 105 of the interleukin-2 sequence (SEQ. ID. NO. I) by a non-reducible surrogate, the stability of the interleukin-2 analogue is considerably better. Although changing the structure of the disulfide bridge can result in the distortion of the tertiary structure, it could be shown that the Cys58-Cys105 bridge of interleukin-2 appears to serve as bridge in an otherwise flexible region of the protein. The interleukin-analogues comprising a non-reducible surrogate at positions 58 or 105 have an intact tertiary structure. Preferably, the non-reducible surrogate is penicillamine (PEN). Most preferably, the synthesis is carried out by Fmoc protected S-acetamidomethyl penicillamine (PEN(Acm)). PEN(Acm) stands for Fmoc-β,β-dimethyl-Cys(Acm)-OH and is commercially available. Such interleukin-2 analogues correspond to SEQ. ID. NO. 2 and SEQ. ID. NO. 3, wherein one of the cysteines at positions 58 and 105 is replaced by PEN, thus forming a more stable and rigid disulfide bond which results in a better overall stability. Coupling of PEN(Acm) is similar to the coupling procedure used for CysAcm, and PEN58Acm is used to replace Cys58Acm.

Another analogue obtained by the method according to the present invention is an interleukin-2 analogue, wherein at position 125 cysteine is replaced by serine (SEQ. ID, NO. 6). Preferably, in addition to the replacement of cysteine at position 125 by serine, one of the cysteines at positions 58 and 105 is replaced by PEN (SEQ. ID. NO. 4 and SEQ. ID. NO. 5). The synthetic route is identical to the method described for the preparation of interleukin-2.

Another analogue obtained by the method according to the present invention is an interleukin-2 analogue, wherein at least one or more of the following amino acid at positions Thr41, Asn71 and Met104 are substituted, and the substitute is preferably homoserine (Hse). Such interleukin-2 analogues correspond to SEQ. ID. NO. 14, SEQ. ID. NO. 15, and SEQ. ID. NO. 16, and. SEQ. ID. NO. 17.

The present invention also encompasses the combination of all variants mentioned above, in particular analogues, wherein one or more of the amino acids at positions Met23, Met39, Met46, Cys58, Cys105, Cys125, Thr41, Asn71 and Met104 are substituted, and the substitutes are
norleucine for Met23, Met39 and/or Met46,
penicillamine for Cys58 or Cys105,
serine for Cys125, and
homoserine for Thr41, Asn71 and Met104.

Especially preferred are interleukin-2 analogues corresponding to SEQ. ID. NO. 18, SEQ. ID. NO. 19, SEQ. ID. NO. 20, SEQ. ID. NO. 21, SEQ. ID. NO. 22, SEQ. ID. NO. 23, SEQ. ID. NO. 24, SEQ. ID. NO. 25, SEQ. ID. NO. 26, SEQ. ID. NO. 27, SEQ. ID. NO. 28, and SEQ. ID. NO. 29.

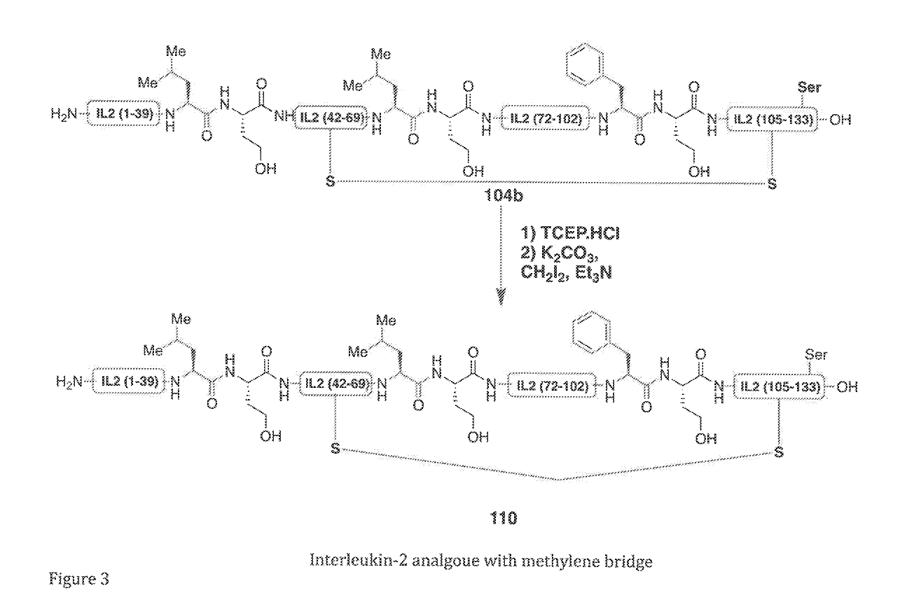

Further, the present invention relates to another analogue containing a methylene thioacetal bridge instead of the Cys58-Cys105 disulfide inter bridge of interleukin-2. It can be produced via a reported protocol (Kourra C. M. B. K.; Cramer, N. Chem Sci. 2016, 7, 7007). The methylene thioacetal is supposed to confer an improved stability to peptides or proteins. The methylene bridge is introduced in the IL-2 after folding. The folded IL-2 is subjected to reducing conditions after which diiodomethane and triethylamine is added to form the methylene thioacetal bridge as shown in FIG. 3.

The at least three, preferably four building blocks used in the method according to the present invention are preferably formed by solid-phase peptide synthesis, preferably by Fmoc-SPPS or Boc-SPPS, most preferably Fmoc-SPPS.

Solid-phase peptide synthesis (SPPS) refers to the direct chemical synthesis of peptides and proteins, wherein an insoluble polymeric support is used as an anchor for the growing protein chain. The free N-terminal amine of a solid-phase attached peptide is coupled to an N-protected amino acid unit. This unit is then deprotected, revealing a new N-terminal amine to which a further amino acid unit may be attached. The general principle of SPPS is that of repeated cycles of such coupling-wash-deprotection-wash steps, adding, typically, one amino acid at a time, until the protein of the desired sequence and length has been synthesized. As understood by those skilled in the art it is possible, in principle, to couple N-protected dipeptides instead of single amino acids to the growing chain in one or more elongation cycles. The present invention also encompasses methods wherein N-protected dipeptides are added to the growing chain.

Preferably, the amino acid residues are anchored to the resin or resin handle through the terminal carboxyl group.

For SPPS, the solid phase is typically a solid, non-soluble support material. Polymeric organic resin supports are the most common type of solid phase material, typically comprising highly solvated polymers with an equal distribution of functional groups. Examples include polystyrene (PS), polyacrylamide (PA), polyethylene glycol (PEG), PEG-polystyrene (PEG-PS) or PEG-polyacrylamide (PEG-PA), and other PEG-based supports.

Suitable materials include but are not limited to: 2-chlorotrityl resin, PEG-HMPB (cross-linked PEG functionalized with 4-(4-hydroxymethyl-3-methoxyphenoxy)butyric acid), Rink amide resin (4-(2′,4′-dimethoxyphenyl-Fmoc-aminomethyl-phenoxy-resin) and Merrifield resin (copolymer of styrene and chloromethylstyrene cross-linked with divinylbenzene). Solid support materials should meet several requirements, besides being chemically inert and able to withstand the conditions of synthesis. That is, for example, solid phase particles are preferably of conventional and uniform size, mechanically robust, easily filterable and highly accessible to the solvents allowing the penetration of the reagents and the enlargement of the peptide chain within its microstructure. Resins as used in the present invention are typically of standard mesh size, which is about 50 to 500 mesh, more preferably 100 to 400 mesh.

Preferably, the hydrophobic building block on the C-terminal region of interleukin-2, preferably building block 4, is prepared on a 2-chlorotrityl resin. The other buiding blocks, preferably building blocks 1, 2 and 3, are preferably prepared on a polyethylene glycol resin (Rink Amide ChemMatrix®) which proved to give a much higher recovery compared to the standard polystyrene resin (Rink Amide polystyrene resin).

Preferably, Fmoc (Fluorenylmethyloxycarbonyl) N-protected amino acids are added to the growing chain. Fmoc protection in solid phase peptide synthesis has significant advantages because its removal involves very mild basic conditions (e.g. piperidine solution), such that it does not disturb the acid labile linker between the peptide and the resin. Fmoc N-protected amino acids are commercially available. Furthermore, reactions to produce Fmoc N-protected amino acids or peptides are common general knowledge for those skilled in the art.

Each incoming amino acid that is added to the growing peptide chain is preferably also protected, where suitable, with a side-chain protecting group, which is typically acid-labile. Protection groups suitable for this purpose are well known in the art. Amino acid residues prone to epimerization such as cysteine and histidine are preferably coupled using preformed 6-Cl-HOBt esters. In a typical procedure, Fmoc-Cys(Trt)-OH or FMoc-Cys(Acm) —OH or Fmoc-His(Trt)-OH (5 equiv relative to resin loading) can be dissolved in a minimal amount of dichloromethane and 6-Cl-HOBt (for example 5.0 equiv) and DIC (for example 5.0 equiv). After stirring for about 30 minutes at room temperature, the solvent can be removed under reduced pressure. Afterwards, a minimal amount of DMF can be added to the resin in order to dissolve the residue and then the reaction is carried out for 2 hours.

Coupling reagents for Fmoc peptide synthesis are well-known in the art. Coupling reagents may be phosphonium salt derivatives of benzotriazole, mixed anhydrides, (e.g. propane phosphonic acid anhydride or ‘T3P’) or other acylating agents such as activated esters or acid halogenides (e.g. isobutyl-chloroformiate or ‘ICBFF’), or they may be carbodiimides (e.g. 1-ethyl-3-(3-dimethylaminopropyl)-carbodiimide, diisopropyl-carbodiimide, dicylcohexyl-carbodiimide), activated benzotriazine-derivatives (e.g. 3-(diethoxypho.,horyloxy)-1,2,3-benzotriazine-4(3H)-one or ‘DEPET’) or uranium. In view of best yield, short reaction time and protection against racemization during chaing elongation, it is preferred that the coupling reagent is selected from the group consisting of uranium salts and phosphonium salts of benzotriazole capable of activating a free carboxylic acid function along with that the reaction is carried out in the presence of a base. Suitable and likewise preferred examples of such uranium or phosphonium coupling salts are e.g. HBTU (0-1H-benzotriazole-1-yl)-N,N,N′,N′-tetramethyluronium hexafluorophosphate), BOP (benzotriazole-1-yl-oxy-tris-(dimethylamino)-phosphonium hexafluorophosphate), PyBOP (Benzotriazole-1-yl-oxy-tripyrrolidinophosphonium hexafluorophosphate), PyBOP, HCTU (0-(1H-6-chloro-benzotriazole-1-yl)-1,1,3,3-tetramethyluronium hexafluorophosphate), TCTU (0-1H-6-chlorobenzotriazole-1-yl)-1,1,3,3-tetramethyluronium tetrafluoroborate), HATU (0-(7-azabenzotriazol-1-yl)-1,1,3,3-tetramethyluronium hexafluorophosphate), TATU (0-(7-azabenzotriazol-1-yl)-1,1,3,3-tetramethyluronium tetrafluoroborate), TOTU (0-[cyano(ethoxy-carbonyl)methyleneamino]-N,N,N N″-tetramethyluronium tetrafluoroborate), HAPyU (0-(benzotriazol-1-yl)oxybis-(pyrrolidino)-uronium hexafluarophosphate. Preferably, the coupling reagent is HCTU.

Activation of the Fmoc amino acid is typically done in the presence of a base reagent. Preferably, the base reagent is a weak base whose conjugated acid has a pKa value of from pKa 7.5 to 15, more preferably of from pKa 7.5 to 10, and which base preferably is a tertiary, sterically hindered amine. Examples of such and further preferred are Hunig-base (N,N-diisopropylethylamine; DIPEA), N,N′-dialkylaniline, 2,4,6-trialkylpyridine, 2,6-trialkylpyridine or N-alkyl-morpholine with the alkyi being straight or branched C1-C4 alkyl, more preferably it is N-methylmorpholine (NMM) or collidine (2,4,6-trimethylpyridine), most preferably N-methylmorpholine (NMM).

The amount of the various reactants in the coupling reaction can vary greatly. Reagents are typically used in large excess to speed-up the reaction and drive it to completion. Typically, the amount of solid support to the amount of Fmoc-amino acid will be a molar ratio ranging from 1:1 to 1:10.

The reaction conditions for the solid phase peptide synthesis, such as reaction time, temperature, and pH may vary without departing from the scope of the invention. The coupling temperature is usually in the range of from 15 to 30° C., preferably at a temperature of about 20 to 25°. Preferably, the coupling is carried out twice (double coupling) in order to increase the yield. Typically, a washing step has to be carried out such as LiCl washes (for example 0.8 M LiCl in DMF) before Fmoc deprotection and coupling of next amino acid. After coupling, unreacted free amine can preferably be capped, for example by treatment with 20% acetic anhydride and 10% NMM (v/v) in DMF for 2×5 min.

In order to prepare a suitable building block for the KAHA ligation, the last amino acid, i.e. the N-terminal amino acid of the building block formed on the resin, has to be linked to a cyclic hydroxylamine. Therefore, after completion of the automated. Fmoc. SPPS, preferably S-N-Boc-5-oxaproline or S-N-Fmoc-oxaproline are introduced by coupling in a separate, non-automated step following procedures known in the art. Such a standard protocol is shown in the below scheme:

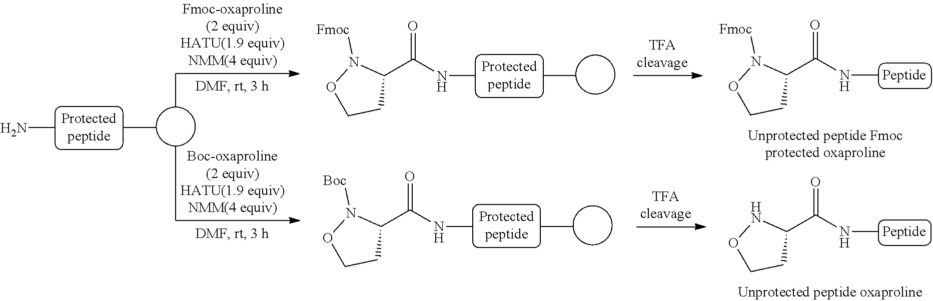

The α-ketoacid group can be obtained for example directly from the protected α-ketoacid monomer or generated by Oxone oxidation of the sulfur glide linker. Further, a linker for the solid phase synthesis of C-terminal α-ketoacids may be obtained by the preparation and oxidation of side-chain unprotected cyanosulfurylides. Upon resin cleavage with TFA, the C-terminal cyanosulfurylide is isolated. It may be oxidized to the aαketoacid by treatment with aqueous, acidic Oxone® for 5 min. In addition, a protecting group for α-ketoacids can be prepared that allows the inclusion of all canonical amino acids, including cysteine, methionine and tryptophan in SPPS and delivers the C-terminal peptide α-ketoacid directly upon cleavage of the resin. Possible protocolls are indicated below:

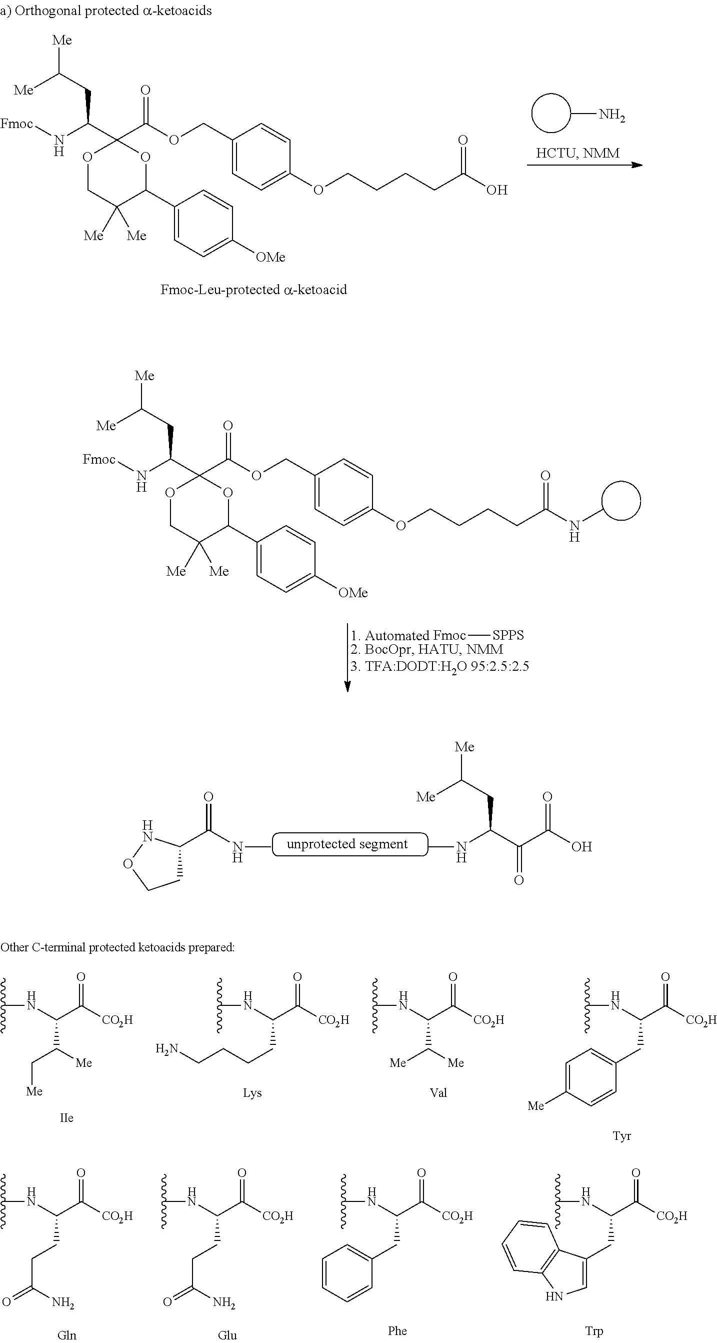

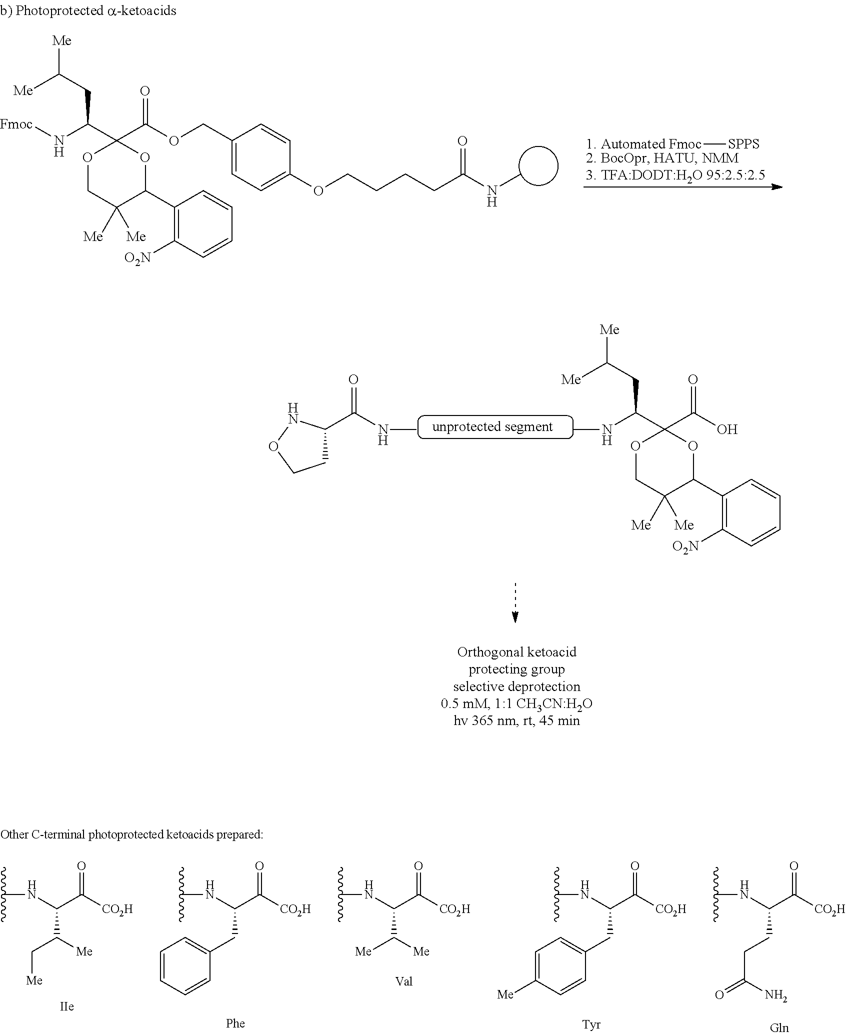

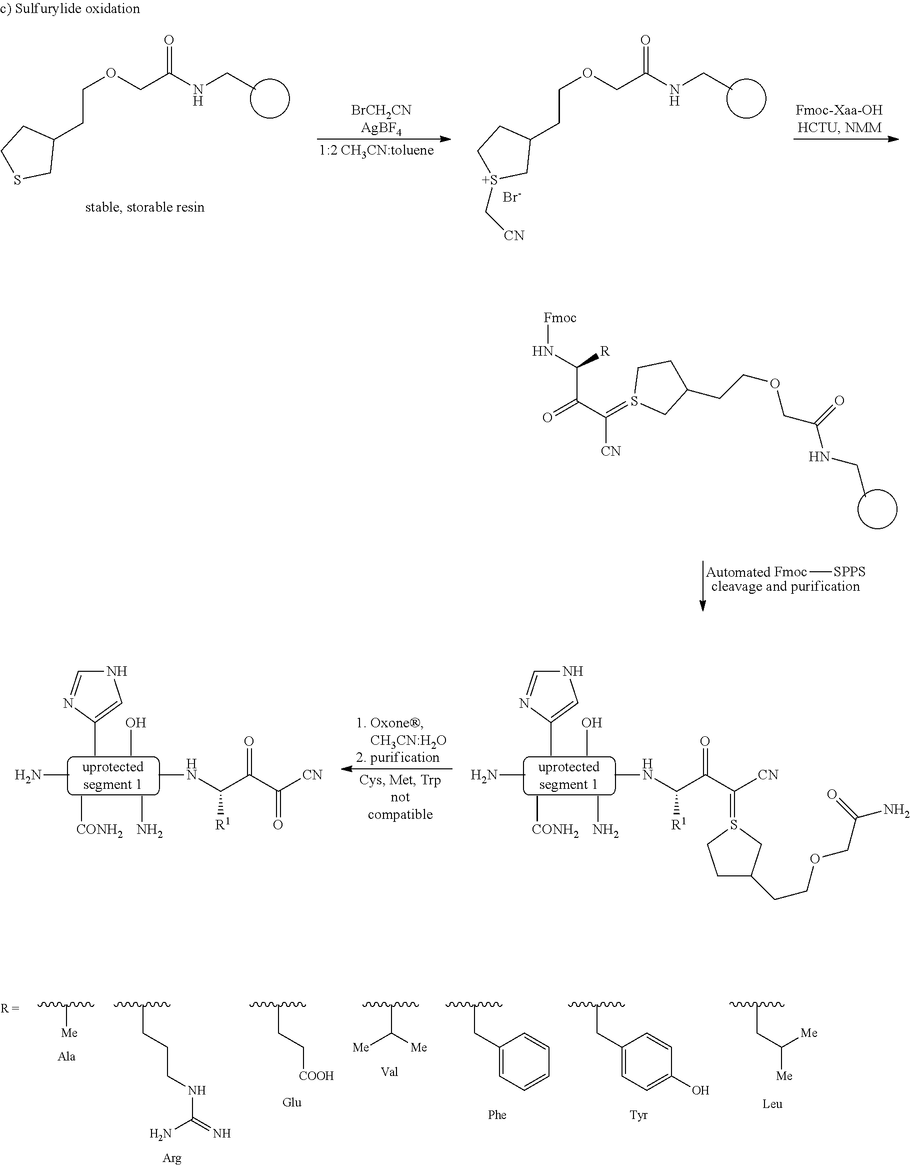

After finishing the synthesis of the building blocks, they are cleaved from resin, preferably with a mixture of 95:2.5:2.5 TFA:DODT (3,6-dioxa-1,8-octanetithiol):water for 2 hours. Preferably, the volume of the solvent is reduced by vaccuum, the crude precipitated in diethylether, centrifugated, decanted and dissolved in a suitable solvent such as DMSO (in particular preferred for building blocks 3 and 4) or 1:1 acetonitrile water 0.1% TFA (for building blocks 1 and 2) for RP-HPLC purification.

Further, the synthetic yield of the building blocks, in particular of building blocks 3 and 4, can be highly s increased by preheating the column to 60° C. before purification.

The building blocks used in the method according to the present invention can be prepared on more than 100 mg scale. The synthesis shown in FIG. 1b forming interleukin-2 with four building blocks by using Fmoc SPPS and assembling via KAHA ligations followed by cysteine deprotection results in over 50 mg of the linear protein with 33% yield overall over KAHA ligation.

Preferably, a mixture of water and an organic solvent is used for the KAHA ligation. Most preferably, the reaction is carried out in a H₂O/DMSO (dimethylsulfoxide) or H₂O/NMP (N-methyl-2-pyrrolidone) mixture at a pH of 3 or less (for example by using aqueous oxalic acid) and at a temperature of about 60° C. Such a solvent system allows a better solubility of the hydrophobic building blocks.

Without any purification, the ligation mixture can be subjected to a one-pot Fmoc deprotection using 10% diethylamine in DMSO for 7 minutes to yield 80 mg of the desired product (building block 3-4) in 53% isolated yield.

Ligation between building block 1 and building block 2 proceedes with complete consumption of the limiting starting material in less than 12 hours.

The crude mixture containing the ligated product can preferably directly subjected to UV irradiation at 365 nm since building block 2 contains a photolabile orthogonal protected group on the ketoacid (C-terminus) to deliver 120 mg of the product (building block 1-2) in an overall yield of 58%.

The final ligation between building block 1-2 and building block 3-4 can be performed, for example, at 15 mM. The desired ligated depsipeptide is cleanly formed after about 10 hours to afford the linear, preferably Cys-protected, interleukin-2 (building block 1-2-3-4) in more than 50% isolated yield in high purity. The method according to the present invention is very effective in the assembling of the segments for interleukin-2 or interleukin-2 analogues due to the use of a mixture of organic solvent and water at acidic pH.

Preferably, the building blocks are dissolved in a minimal amount of DMSO or NMP and 0.1 M aqueous oxalic acid and warmed to 60° C. in order to carry out the ligation. The ligations can be carried out independently of the scale with even more than 300 mg of building blocks. The concentration and the reaction time are preferably between 10 and 20 mM and 8 and 16 hours.

The method according to the present invention is very effective for the preparation of interleukin-2 or interleukin-2 analogues due to the generation of depsipeptides at the ligation sites. The depsipeptides are more polar and more soluble than their amide counterparts. Both results in a higher solubility of the building blocks, and thus result in a higher yield. The depsipeptides can readily be rearranged to the corresponding amides in basic buffers at a pH ranging from 8 to 10.

The folding procedure is known to the skilled person, for example the conditions mentioned in Asashina, Y. et al., Chemical Synthesis of O-Glycosylated Human Interleukin-2 by the Reverse Polarity Protection Strategy, Angew. Chem. Int. Ed. 2015, 54, 8226-8230 can be followed.

The purity of the final protein can be confirmed by Reversed Phase HPLC (RP-HPLC) and matrix assisted laser desorption ionization-time of flight mass spectrometry (MALDI-TCP) and SDS-PAGE (sodium dodecyl sulfate polyacrylamide gel electrophoresis). The biological activity can be confirmed, for example, by cell proliferation assays using cytotoxic T-cell line (CTLL-2) bioassay. CTLL-2 cells respond specifically to human IL-2 and a dose-response curve using synthetic IL-2 and recombinant IL-2 is constructed to determine the activity (Davis, L. S.; Lipsky, P. E.; Bottomly, K. Current Protocols in Immunology 2001).

The present invention also relates to the interleukin-2 analogues obtained by the method according to the present invention. Said interleukin-2 analogues are preferably selected from the group consisting of SEQ. ID. No. 2, SEQ. ID. No. 3, SEQ. ID. No. 4, SEQ. ID. No. 5, SEQ. ID. No. 6, SEQ. ID. No. 7, SEQ. ID. No. 8, SEQ. ID. No. 9, SEQ. ID. No, 10, SEQ. ID. No. 11, SEQ. ID. No. 12, SEQ. ID. No. 13, SEQ. ID. No. 14, SEQ. ID. No. 15, SEQ. ID. No. 16, SEQ. ID. No. 17, SEQ. ID. No. 18, SEQ. ID. No. 19, SEQ. ID. No. 20, SEQ. ID. No. 21, SEQ. ID. No. 22, SEQ. ID. No, 23, SEQ. ID. No. 24, SEQ. ID. No. 25, SEQ. ID. No, 26, SEQ. ID. No. 27, SEQ. ID. No. 28, and SEQ. ID. No. 29. Said analogues have an improved stability,

The present invention relates further to the building s blocks which allow the fast and efficient synthesis of interleukin-2 or interleukin-2 analogues according to the present invention. Particular preferred building blocks are selected from the group consisting of SEQ. ID. No. 30, SEQ. ID. No. 31, and SEQ. ID. No. 32, SEQ. ID. No. 33, SEQ. ID. No. 34, SEQ. ID. No. 35, SEQ. ID. No. 36, and SEQ. ID. No. 37.

The present invention relates further to a composition comprising interleukin-2 or at least one in terleukin-2 analogue obtained according to the method of the present invention. Such a composition is therapeutically active for use in the treatment of cancer and for use in the treatment of infectious diseases. In particular, the present invention also relates to a composition for use in the treatment of tumors in human or animal organisms and for use in the immunization of human or animal organisms against this tumor, said composition comprising a synergistic association of: cells, viruses or bacteria transiently expressing in the organism at least one gene enabling them to produce in vivo one or more immunomodulators, and viruses, or cells producing viruses, said viruses if possible preferably infecting dividing cells of the treated organisms and carrying within their genome at least one gene whose expression in the dividing cells will cause their destruction. Preferably, the composition comprises additionally polyols, sugars or polymers such as polyethylene glycol in order to the effectiveness of interleukin. Preferably, such a composition is administered intravenous.

| SEQ ID. | |
|---|---|
| NO. | Interleukin-2 or Interleukin-2 analogue |
| 1 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 2 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln PEN Leu Glu |
| | Glu Glu Leu Lys Pro Leu Gln Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 3 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met PEN Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 4 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln PEN Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 3 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Gln Leu Lys Gly Ser Gln |
| | Thr Thr Phe Met PEN Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 6 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln Cys Leu Gln |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 7 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 8 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Nle Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 9 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 10 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 11 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Met Leu |
| | Thr Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 12 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Nle Leu |
| | Thr Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 13 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Thr Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 14 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Hse Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 15 | Ala Pro thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 16 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Thr Phe Lys Phe Tyr Met Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 17 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Hse Phe Lys Phe Tyr Met Pro Lys Lys Ala Thr Glu Leu Lys His Leu Gln Cys Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 18 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Thr Phe Lys Phe Tyr Nle Pro Lys Lys Ala Thr Glu Leu Lys His Leu Gln PEN Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 19 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Thr Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met PEN Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 20 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Thr Phe Lys Phe Tyr Nle Pro Lys Lys Ala Thr Glu Leu Lys His Leu Gln PEN Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met Cys Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 21 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Thr Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Asn Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Met PEN Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 22 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Hse Phe Lys Phe Tyr Met Pro Lys Lys Ala Thr Glu Leu Lys His Leu Gln PEN Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 23 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Hse Phe Lys Phe Tyr Met Pro Lys Lys Ala Thr Glu Leu Lys His Leu Gln Cys Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse PEN Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 24 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Hse Phe Lys Phe Tyr Met Pro Lys Lys Ala Thr Glu Leu Lys His Leu Gln PEN Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse Cys Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 25 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Met Leu |
| | Hse Phe Lys Phe Tyr Met Pro Lys Lys Ala Thr Glu Leu Lys His Leu Gln Cys Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse PEN Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 26 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Hse Phe Lys Phe Tyr Nle Pro Lys Lys Ala Thr Glu Leu Lys His Leu Gln PEN Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse Cys Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 27 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Hse Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse PEN Glu Tyr Ala Asp Glu Thr Ala Thr Ile Val Glu Phe Leu Asn Arg |
| | Trp Ile Thr Phe Cys Gln Ser Ile Ile Ser Thr Leu Thr |
| 28 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Hse Phe Lys Phe Tyr Nle Pro Lys Lys Ala Thr Glu Leu Lys His Leu Gln PEN Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse Cys Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 29 | Ala Pro Thr Ser Ser Ser Thr Lys Lys Thr Gln Leu Gln Leu Glu His Leu Leu Leu Asp |
| | Leu Gln Nle Ile Leu Asn Gly Ile Asn Asn Tyr Lys Asn Pro Lys Leu Thr Arg Nle Leu |
| | Hse Phe Lys Phe Tyr Nle Pro Lys Lys Ala Thr Glu Leu Lys His Leu Gln Cys Leu Glu |
| | Glu Glu Leu Lys Pro Leu Glu Glu Val Leu Hse Leu Ala Gln Ser Lys Asn Phe His Leu |
| | Arg Pro Arg Asp Leu Ile Ser Asn Ile Asn Val Ile Val Leu Glu Leu Lys Gly Ser Glu |
| | Thr Thr Phe Hse PEN Glu Tyr Ala Asp Glu Thr Ala Thr Ile Val Glu Phe Leu Asn Arg |
| | Trp Ile Thr Phe Ser Gln Ser Ile Ile Ser Thr Leu Thr |
| 30 | Hse Cys Glu Tyr Ala Asp Glu Thr Ala Thr Ile Val Glu Phe Leu Asn Arg Trp Ile Thr |
| | Phe Ser Gln Ser Ile Ile Ser Thr Leu Thr |
| 31 | Ala Pro Thr Ser Ser Ser Thr Lys Lys Thr Gln Leu Gln Leu Glu His Leu Leu Leu Asp |
| | Leu Gln Met Ile Leu Asn Gly Ile Asn Asn Tyr Lys Asn Pro Lys Leu Thr Arg Nle Leu |
| 32 | Hse Phe Lys Phe Tyr Nle Pro Lys Lys Ala Thr Glu Leu Lys His Leu Gln Cys Leu Glu |
| | Glu Glu Leu Lys Pro Leu Glu Glu Val Leu |
| 33 | Hse Leu Ala Gln Ser Lys Asn Phe His Leu Arg Pro Arg Asp Leu Ile Ser Asn Ile Asn |
| | Val Ile Val Leu Glu Leu Lys Gly Ser Glu Thr Thr Phe |
| 34 | Hse Cys Glu Tyr Ala Asp Glu Thr Ala Thr Ile Val Glu Phe Leu Asn Arg Trp Ile Thr |
| | Phe Cys Gln Ser Ile Ile Ser Thr Leu Thr |
| 35 | Hse Phe Lys Phe Tyr Nle Pro Lys Lys Ala Thr Glu Leu Lys His Leu Gln PEN Leu Glu |
| | Glu Glu Leu Lys Pro Leu Glu Glu Val Leu |
| 36 | Hse PEN Glu Tyr Ala Asp Glu Thr Ala Thr Ile Val Glu Phe Leu Asn Arg Trp Ile Thr |
| | Phe Cys Gln Ser Ile Ile Ser Thr Leu Thr |
| 37 | Hse PEN Glu Tyr Ala Asp Glu Thr Ala Thr Ile Val Glu Phe Leu Asn Arg Trp Ile Thr |
| | Phe Ser Gln Ser Ile Ile Ser Thr Leu Thr |

### EXAMPLES

General Methods

HPLC

Peptides and protein segments were analyzed and purified by reversed phase high performance liquid chromatography (RP-HPLC) on Jasco analytical and preparative instruments equipped with dual pumps, mixer and in-line degasser, a variable wavelength UV detector (simultaneous monitoring of the eluent at 220 nm, 254 nm and 301 nm) and a Rheodyne injector fitted with a 20 μl, 500 μl or 1000 μl, 5 mL or 20 mL injection loop or on a Gilson preparative instrument fitted with a 10 mL injection loop. If required, the columns were preheated using an Alltech column heater or a water bath (preparative HPLC). The mobile phase for RP-HPLC were Milipore-H₂O containing 0.1% TFA and HPLC grade CH₃CN containing 0.1% TFA. In the described HPLC analysis and purifications, TFA was always used as solvent modifier. Analytical HPLC was performed on a Shiseido Capcell Pak UG80 C18 UG120 (5 μm, 120 Å pore size, 4.6 mm I.D.×250 mm) column, on a Shiseido Capcell Pak UG80 C18 UG 80 (5 μm, 120 Å pore size, 4.6 mm I.D.×250 mm) column or on a Shiseido MGII C18 column (5 μm, 4.6 mm I.D.×250 mm) columns at a flow rate of 1 mL/min. Preparative HPLC was performed on a Shiseido Capcell Pak MGII column (5 μm, 100 Å pore size, 20 mm I.D.×250 mm), on Shiseido Capcell Pak C4 or UG80 C18 columns (5 μm, 80 Å pore size, 50×250 mm) or on a Phenomenex Jupiter C4 column (5 μm, 300 Å pore size, 30 mm I.D.×250 mm) at indicated flow rates (typically 10 or 40 mL/min).

The following type of method was used: the column was pre-equilibrated at starting solvent composition for typically 3-7 min. After injection of the sample, the solvent composition was un to the final solvent composition (e.g. 50% CH₃CN). After the gradient run time, the solvent composition was changed to 95% CH₃CN within 1 min and the column was flushed for 5-7 min. Within 1 min, the solvent composition was changed to 10% CH₃CN and the run ended. For the sake of simplicity, only the gradient time and the starting and end composition of the eluent will be stated at the individual experiments, although all experiments included the full cycle as described above.

Solid Phase Peptide Synthesis (SPPS)

Peptides were synthesized on a CS Bio 136X synthesizer using Fmoc-SPPS chemistry. The following Fmoc-amino acids with side-chain protection groups were used: Fmoc-Ala-OH, Fmoc-Arg(Pbf)-OH, Fmoc-Asn(Trt)-OH, Fmoc-Asp(OtBu)-OH, Ftoc-Cys(Acm)-OH, Fmoc-Gln(Trt)-OH, Fmoc-Glu(OtBu)-OH, Fmoc-Gly-OH, Fmoc-His(1-Trt)-OH, Ftoc-Ile-OH, Fmoc-Leu-OH, Fmoc-Lys(Boc)-OH, Fmoc-Met-OH, Fmoc-Phe-OH, Fmoc-Pro-OH, Fmoc-Ser(tBu)-OH, Fmoc-Thr(tBu)-OH, Fmoc-Trp(Boc)-OH, Fmoc-Tyr(tBu)-OH, Fmoc-Val-OH. SPPS was performed on Rink-amide polystyrene resin, Rink-amide ChemMatrix resin, Wang polystyrene resin or 2-chlorotrityl polystyrene resin.

Manual loading of the first amino acid residue on the resin and subsequent Fmoc-SPPS, followed established standard protocols. A brief summary of the utilized synthesis protocols: Fmoc-deprotections were performed with 20% piperidine in DMF (2×8 min). Couplings were is performed with Fmoc-amino acid (4.0 equiv relative to resin substitution), HCTU (3.9 equiv) and NMM (8.0 equiv) in DMF for GO min. If required, the coupling step was repeated once (double coupling) and LiCl washes (0.8 M LiCl in DMF) were performed before Fmoc-deprotection and coupling. After coupling, unreacted free amine was capped by treatment with 20% acetic anhydride and 10% NMM in DMF for 2×5 min.

Amino acid residues prone to epimerization such as cysteine were coupled using preformed 6-Cl-HOBt esters, In a typical procedure, Fmoc-Cys(Acm)-OH (5.0 equiv relative to resin loading) was dissolved in a minimal amount of CH₂Cl₂, and 6-Cl-HOBt (5.0 equiv) and DIC (5.0 equiv) were added. The mixture was stirred for 15 min at rt, the solvent concentrated under reduced pressure and the residue dissolved in a minimal amount of DMF, added to the resin and allowed to react for 2 h.

Manual Coupling of Special Amino Acids

Valuable non-standard monomers (e.g. protected 5-oxaproline: BocOpr, FmocOpr) were coupled manually. The monomer (2.5 equiv) was dissolved in a minimal amount of DMF (minimal concentration of monomer: 0.1 M), HATU (2.48 equiv) and NMM (5 equiv) were added. After a brief period of preactivation (2 min), the solution was added to the resin and allowed to react for 2 h.

Resin Cleavage Procedures

Method A: General cleavage protocol for peptide segments synthesized on Rink-amide polystyrene resin or 2-chlorotrityl polystyrene resin. The dry resin was placed in a glass vial, a mixture of 95:2.5:2.5 TFA:TIPS:H₂O (15 mL(g resin) was added and the suspension shaken for 2 h. The resin was removed by filtration and washed with TFA (5 mL/g resin), the filtrate was placed in a plastic centrifugal tube (40 mL) and volatiles removed under reduced pressure. The residue was triturated with Et₂O (ca. 15 mL/g resin), centrifuged (2500 ×g, 4 min) and the supernatant was removed by decantation. This trituration/washing step was repeated once. The crude material was dried and dissolved in a suitable solvent (DMSO or 1:1 CH₃CN:H₂O+0.1% TFA) for RP-HPLC purification.

Method B: Cleavage protocol for peptide α-ketoacid segments synthesized on α-ketoacid resins. The dry resin was placed in a glass vial, a mixture of 95:2.5:2.5 TFA:DODT:H₂O (15 mL/g resin) was added and the suspension shaken for 1.5 h. The resin was removed by filtration and washed with TFA (5 mL/g resin), the filtrate was placed in a plastic centrifugal tube (40 mL) and volatiles removed under reduced pressure. The residue was triturated with Et₂O (ca. 15 mL/g resin), centrifuged (500 ×g, 4 min) and the supernatant was removed by decantation. This trituration/washing step was repeated once. The crude material was dried and dissolved in a suitable solvent (DMSO or 1:1 CH₃CN:H₂O+0.1% TFA) for RP-HPLC purification.

Synthesis of Interleukin-2

FIG. 1a) shows the amino acid sequence of interleukin-2 together with the building blocks.

FIG. 1b) shows the synthesis of interleukin

1.1.Synthesis of Protein Segments

Building block 1: Synthesis of IL2(1-39)-Leu-α-Ketoacid 90

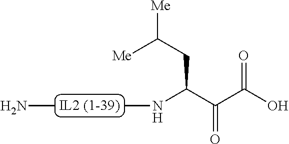

NH₂-IL2(1-39)-Leu-α-ketoacid 90 was synthesized on Rink-Amide Chemmatrix resin (1.0 g, 0.56 mmol/g) preloaded with protected Fmoc-Leu-α-ketoacid with a substitution capacity of 0.15 mmol/g. The synthesis was performed on 0.15 mmol scale (1.0 g of resin, 1.0 equiv) by automated Fmoc-SPPS up to Ala1 using the procedure described the General Methods. Fmoc-Nle-OH was used to replace of Met23 and Met39. The peptide was cleaved from resin following Method B. The crude peptide was dissolved in a mixture of CH₃CN:H₂O (+0.1% TFA) and purification of crude NH₂-IL2(1-39)-Leu-α-ketoacid 90 was performed by preparative HPLC using Shiseido Capcell Pak UG80 C18 column (50×250 mm) with a gradient of 25 to 60% CH₃CN with 0.1% TFA in 30 min, flow rate 40 mL/min. The pure product fractions were pooled and lyophilized to obtain NH₂-IL2(1-39)-Leu-α-ketoacid 90 (182 mg, 39.9 μmol, 26.6% yield for peptide synthesis, resin cleavage and purification steps). Analytical HPLC and MALDI FTMS were used to confirm the purity and exact mass of the product 90. m/z calculated for C₂₀₄H₃₄₇N₅₆O₆₁ [M+H]⁺: 4559.5780; measured 4559.5824.

Building Block 2: Synthesis of Opr-IL2(42-69)-Photoprotected-Leu-α-Ketoacid 98

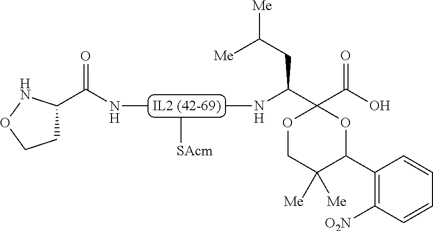

Opr-IL2 (42-69)-Photoprotected-Leu-α-ketoacid 98 was synthesized on Rink-Amide Chemmatrix resin (1.0 g, 0.56 mmol/g) preloaded with Photoprotected Fmoc-Leu-α-ketoacid with a determined loading of 0.15 mmol/g. The synthesis was performed on 0.15 mmol scale (1.0 g of resin, 1.0 equiv) by automated Fmoc-SPPS up to Phe42 using the procedure described in the General Methods. Fmoc-Cys(Acm)-OH was used for the coupling of Cys58. Fmoc-Nle-OH was used to replace of Met46. After automated Fmoc-SPPS, BocOpr (65 mg, 0.3 mmol, 2.0 equiv to resin) was coupled to the free amine on-resin using HATU (111 mg, 0.29 mmol, 1.95 equiv to resin) and NMM (65 μL, 0.6 mmol, 4.0 equiv to resin) for 2 h. The peptide was cleaved from resin following Method B. The crude peptide was dissolved in CH₃CN:H₂O+0.1% TFA and purification of crude Opr-IL2(42-69)-Photoprotected-Leu-α-ketoacid 98 was performed by preparative HPLC using Shiseido Capcell Pak UG80 C18 column (50×250 mm) with a gradient of 25 to 60% CH₃CN with 0.1% TFA in 30 min, flow rate 40 mL/min. The pure product fractions were pooled and lyophilized to obtain Opr-IL2(42-69)-Photoprotected-Leu-α-ketoacid 98 (173 mg, 44.0 μmol, 29.4% yield for peptide synthesis, resin cleavage and purification steps). Analytical HPLC and MALDI FTMS were used to confirm the purity and exact mass of the product 98. m/z calculated for C₁₈₄H₂₈₆NaN₄₀O₅₂S [M+Na]⁺: 3945.0621; measured 3945.0642.

Building Block 3: Synthesis of Fmoc IL2(72-102)-Phe-α-Ketoacid 99

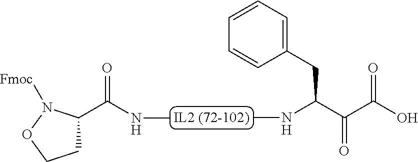

FmocOpr-IL2(72-102)-Phe-α-ketoacid 99 was synthesized on Rink-Amide Chemmatrix (1.0 g, 0.56 mmol/g) resin preloaded with protected Fmoc-Phe-α-ketoacid with a determined loading of 0.21 mmol/g. The synthesis was performed on 0.21 mmol scale (1.0 g of resin, 1.0 equiv) by automated Fmoc-SPPS up to Leu73 using the procedure described in the General Methods. Double couplings were used after each coupling and cappings were also added after following residues: Trp, Asn, His, Leu, Ile, Val, Arg, Thr, Asn, Phe, Tyr. At the end of the synthesis, FmocOpr (104 mg, 0.30 mmol, 2.00 equiv to resin) was coupled to the free amine on-resin using HATU (111 mg, 0.29 mmol, 1.95 equiv to resin) and NMM (65 μL, 0.6 mmol, 4.0 equiv to resin) for 2 h. The peptide was cleaved from resin following Method B. The crude peptide was dissolved in DMSO and purification of crude FmocOpr-IL2(72-102)-Phe-α-ketoacid was performed by preparative HPLC using Shiseido Capcell Pak UG80 C18 column (50×250 mm) preheated to 60° C., with a gradient of 30 to 60% CH₃CN with 0.1% TFA in 30 min, flow rate 40 mL/min. The pure product fractions were pooled and lyophilized to obtain FmocOpr-IL2(72-102)-Phe-α-ketoacid 99 (97.0 mg, 24.2 μmol, 12.1% yield for peptide synthesis, resin cleavage and purification steps). Analytical HPLC and MALDI FTMS were used to confirm the purity and exact mass of the product 99. m/z calculated for C₁₈₄H₂₈₅N₄₇O₅₃ [M+H]⁺: 4002.11236; measured 4002.10446.

Building Block 4: Synthesis of Opr-IL2(105-133)-Acm 100a

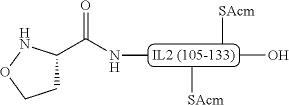

Opr-IL2(105-133)-Acm 100a was synthesized on 2-chlorotrityl polystyrene resin preloaded with Fmoc-Thr(OtBu)-OH and a loading of 0.25 mmol/g. The synthesis was performed on 0.25 mmol scale (1.0 g of resin, 1.0 equiv) by automated Fmoc-SPPS up to Glu106 using the procedure described in the General Methods. Double couplings were used after each coupling and cappings were also added after following residues: Trp, Asn, His, Leu, Ile, Val, Arg, Thr, Asn, Phe, Tyr. Fmoc-Cys(Acm)-OH was used for the coupling of Cys125 and Cys105. Isoacyldipeptide Ile129-Ser130 and pseudoproline Ala112-Thr113 were used for optimization of the peptide synthesis. After automated Fmoc-SPPS, BocOpr (108 mg, 0.50 mmol, 2.00 equiv to resin) was coupled to the free amine on-resin using HATU (182 mg, 0.48 mmol, 1.95 equiv to resin) and NMM (108 μL, 1.00 mmol, 4.00 equiv to resin) for 2 h. The peptide was cleaved from resin following Method A. The crude peptide was solved in DMSO and purification of crude Opr-IL2(105-133)-Acm 100a was performed by preparative HPLC using Shiseido Capcell Pak UG80 C18 column (50×250 mm) preheated to 60° C., with a gradient of 40 to 80% CH₃CN with 0.1% TFA in 30 min, flow rate 40 mL/min. The pure product fractions were pooled and lyophilized to obtain Opr-IL2(105-133)-Acm 100a (79 mg, 22 μmol, 8.7% yield for peptide synthesis, resin cleavage and purification steps). Analytical HPLC and MALDI FTMS were used to confirm the purity and exact mass of the product 100a. m/z calculated for C₁₆₁H₂₄₆N₃₈O₅₂S₂ [M+H]⁺: 3607. 209; measured 3607.7288.

Synthesis of Opr-IL2(105-133)-Ser125 100b

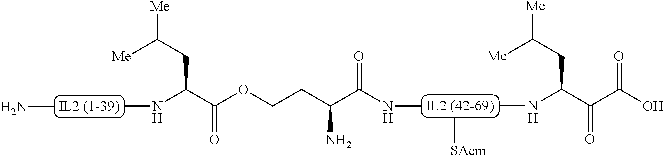

Opr-IL2 (105-133)-Ser125 100b was synthesized on 2-chlorotrityl polystyrene resin preloaded with Fmoc-Thr(OtBu)-OH and a loading of 0.25 mmol/g. The synthesis was performed on 0.25 mmol scale (1.0 g of resin, 1.0 equiv) by automated Fmoc-SPPS up to Cys105 using the procedure described in the General Methods. Double couplings were used after each coupling and cappings were also added after following residue Trp, Asn, His, Leu, Ile, Val, Arg, Thr, Asn, Phe, Tyr. Fmoc-Cys(Acm)-OH was used for the coupling of Cys105 and Fmoc-Ser(OtEu)-OH was used to replace Cys125, Isoacyldipeptide Ile129-Ser130 and pseudoproline Ala112-Thr113 were used for optimization of the peptide synthesis. After automated Fmoc-SPPS, BocOpr (108 mg, 0.50 mmol, 2.00 equiv to resin) was coupled to the free amine on-resin using HATU (140 mg, 0.49 mmol, 1.95 equiv to resin) and NMM (108 μL, 1.00 mmol, 4.00 equiv to resin) for 2 h. The peptide was cleaved from resin following Method A. Purification of crude Opr-IL2(105-133)-Ser125 100b was performed by preparative HPLC using Shiseido Capcell Pak UG80 C18 column (50×250 mm) preheated to 60° C., with a gradient of 40 to 80% CH₃CN with 0.1% TFA in 30 min, flow rate 40 mL/min. The pure product fractions were pooled and lyophilized to obtain Opr-IL2(105-133)-Ser125 100b (76 mg, 21 μmol, 8.61 yield for peptide synthesis, resin cleavage and purification steps). Analytical HPLC and MALDI FTMS were used to confirm the purity and exact mass of the product 100b. m/z calculated for C₁₅₈H₂₄₂N₃₇O₅₂S [M+H]⁺:3521-7145; measured 3521.7149.

1,2. Assembling of Segments to IL-2

Building Block 1-2: Synthesis of NH₂-IL2(1-69)-Leu-α-Ketosoid 102

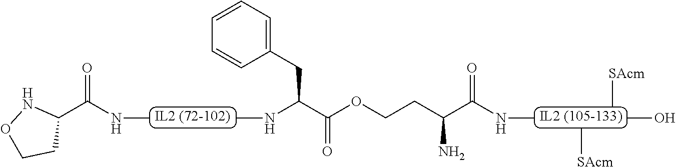

NH₂-IL2 (1-39)-Leu-α-ketoacid 90 (100 mg, 21.9 μmol, 1.30 equiv) and Opr-IL2(42-69)-Photoprotected-Leu-α-ketoacid 98 (66.0 mg, 16.8 μmol, 1.00 equiv) were dissolved in 9:1 DMSO:H₂O with 0.1 M oxalic acid (844 μL, 20 mM) and the solution was shaken at 60° C. The progress of the ligation was monitored by analytical HPLC using a Shiseido Capcell Pak UG80 C18 column (4.6×250 mm) with a gradient of 20 to 95% CH₃CN with 0.1% TFA in 20 min. An aliquot of the ligation mixture (0.1 μL) was taken at various time point, diluted to 12 μL with 1:1 CH₃CN:H₂O and injected on HPLC. After completion of the ligation (16 h), the reaction mixture was diluted to 8.5 mL with 1:1 CH₃CN: H₂O with 0.1% TFA and irradiated at a wavelength of 365 nm for 45 min. The reaction mixture was purified by preparative HPLC using a Shiseido Capcell Pak UG80 C18 column (50×250 mm) with a gradient of 30 to 60% CH₃CN with 0.1% TFA in 30 min, flow rate 40 mL min. The fractions containing the gated product were pooled and lyophilized to give pure NH₂-IL2(1-69)-Leu-α-ketoacid 102 (82 mg, 9.9 μmol, 61% yield for ligation and UV deprotection steps). Analytical HPLC and ESI-HRMS were used to confirm the purity and identity of NH₂-IL2(1-69)-Leu-α-ketoacid 102. m measured for NH₂-IL2 (1-69)-Leu-α-ketoacid 102 C₃₇₆H₆₁₉N₉₅O₁₀₈S [M+H]⁺: 8230,5913.

Building Block 3-4: Synthesis of Opr-IL2(72-133)-Acm 101a

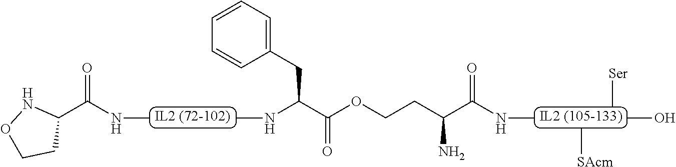

Opr-IL2(105-133)-Ser125 100a (65.0 mg, 17.9 μmol, 1.00 equiv) and FmocOpr-IL2 (72-102)-Phe-α-ketoacid 99 (96.0 mg, 23.3 μmol, 1.30 equiv) were dissolved in 9:1 DMSO: H₂O with 0.1 M oxalic acid (0.9 □L, 20 mM) and the solution was shaken at 60° C. The progress of the ligation was in monitored by analytical HPLC using a Shiseido Capcell Pak UG80 C18 column (4.6×250 mm) with a gradient of 40 to 95% CH₃CN with 0.1% TFA in 20 min. An aliquot of the ligation mixture (0.1 μL) was taken at various time point, diluted to 12 μL with 1:1 CH₃CN:H₂O and injected on HPLC. After completion of the ligation (16 h), the reaction mixture was diluted to 4 mL with DMSO and 200 μL diethylamine was added dropwise and the solution was shaken for 7 min at rt. The reaction mixture was diluted to 10 mL with DMSO and purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure Opr-IL2(72-133)-Acm 101 a (68 mg, 9.2 μmol, 51% yield for ligation and Fmoc-deprotection steps), Analytical HPLC and MALDI FTMS were used to confirm the purity and identity of Opr-IL2 (72-133)-Acm 101a, m/z measured for C₃₂₉H₅₂₀N₈₄O₁₀₁S₂ [M+H]⁺: 7360.8905.

Synthesis of Opr-IL2(72-133)-Ser125 101b

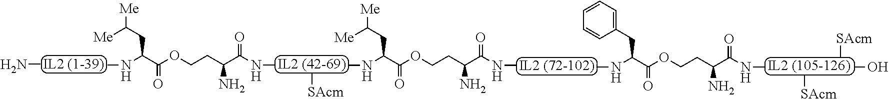

Opr-IL2 (72-133)-Ser125 100b (22 mg, 6.2 μmol, 1.0 equiv) and FmocOpr-IL2(72-102)-Phe-α-ketoacid 99 (33 mg, 8.1 μmol, 1.3 equiv) were dissolved in 9:1 DMSO: H₂O with 0.1 M oxalic acid (311 μL, 20 mM) and the solution was shaken at 60° C. The progress of the ligation was monitored by analytical HPLC using using the same conditions as for Opr-IL2 (72-133)-Acm 100a. After completion of the ligation (16 h), the reaction mixture was diluted to 2 mL with DMSO and 100 μL diethylamine was added dropwise and the solution was shaken for 7 min at rt. The reaction mixture was diluted to 8 mL with DMSO and purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min, The fractions containing the ligated product were pooled and lyophilized to give pure Opr-IL2(72-133)-Ser125 101b (23 mg, 3.1 μmol, 50% yield for ligation and Fmoc-deprotection steps). Analytical HPLC and ESI-HRMS were used to confirm the purity and identity of Opr-IL2 (72-133)-Ser125 101b. m/z measured for C₃₂₆H₅₁₆N₈₄O₁₀₁S [M+H]⁺: 7259.80.

Building Block 1-4: Synthesis of NH₂-IL2(1-133)-Acm 103a

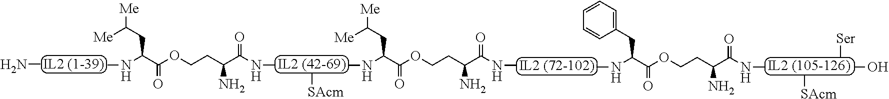

NH₂-IL2(1-69)-Leu-α-ketoacid 102 (80 mg, 9.2 μmol, 1.3 equiv) and. IL2(72-133)-Acm 101a (55 mg, 7.4 μmol, 1.0 equiv) were dissolved in 9:1 DMSO:H₂O with 0.1 M oxalic acid (500 μL, 15 mM) and the solution was shaken at 60° C. The progress of the ligation was monitored by analytical HPLC using a Shiseido Capcell Pak UG80 C18 column (4.6×250 mm) with a gradient of 30 to 80% CH₃CN with 0.1% TFA in 35 min. An aliquot of the ligation mixture (0.1 μL) was taken at various time point, diluted to 12 μL with 1:1 CH₃CN:H₂O and injected on HPLC. After completion of the ligation (16 h), the reaction mixture was diluted to 8 mL with DMSO and purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2(1-133)-Acm 103a (64 mg, 4.1 μmol, 55% yield). Analytical HPLC and ESI-HRMS were used to confirm the purity and identity of NH₂-IL2(1-133)-Acm 103a. m/z measured for C₇₀₄H₁₁₄₀N₁₈₀O₂₀₇S₃ [M+H]⁺: 15533.3742.

Synthesis of NH₂-IL2(1-133)-Ser125 103b

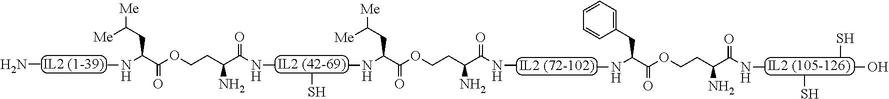

NH₂-IL2(1-69)-Leu-α-ketoacid 102 (44 mg, 5.3 μmol, 1.3 equiv) and IL2(72-133)-Ser125 101b (30 mg, 4.1 μmol, 1.0 equiv) were dissolved in 9:1 DMSO:H₂O with 0.1 M oxalic acid (273 μL, 15 mM) and the solution was shaken at 60° C. The progress of the ligation was monitored by analytical HPLC using the same conditions as for NH₂-IL2(1-133)-Acm 103a. After completion of the ligation (16 h), the reaction mixture was diluted to 8 mL with DMSO and purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2(1-133)-Ser125 103b (33 mg, 4.1 μmol, 52% yield). Analytical HPLC and ESI-HRMS were used to confirm the purity and identity of NH₂-IL2(1-133)-Ser125 103b. m/z calculated for C₇₀₁H₁₁₃₅N₁₇₉O₂₀₇S₂ [M+H]⁺: 15446.348, measured: 15446.3899.

Synthesis of NH₂-11.2(1-133)-SH 107a

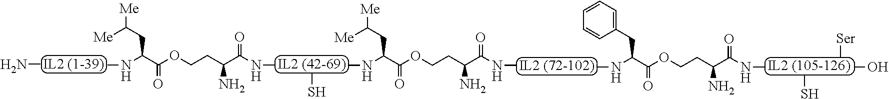

NH₂-IL2(1-133)-Acm 103a (10 mg, 0.6 μmol, 1.0 equiv) was dissolved in a 50% aqueous solution of acetic acid (2.57 mL, 0.25 mM) containing 1% AgOAe then the mixture was vortexed for 2 h at 50° C. in the dark. 50% aqueous solution of acetic acid containing 10% DTT (4.0 ml) was added to the mixture, then the formed precipitate was separated after centrifugation. The precipitate was repeatedly washed with same solution and the combined supernatant (ca. 10 mL) was purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2(1-133)-SH 107a (8.0 mg, 0.5 μmol, 85% yield). Analytical HPLC and ESI-HRMS were used to confirm the purity and identity of NH₂-IL2(1-133)-SH 107a, m/z measured for C₆₉₅H₁₁₂₅N₁₇₇O₂₀₄S₃ [M+H]⁺: 15320.2823.

Synthesis of NH₂-IL2(1-133)-Ser125-SH 107b

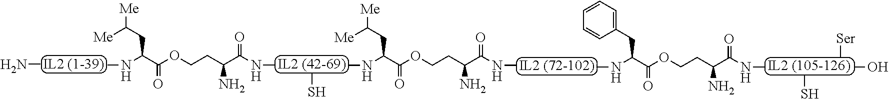

NH₂-IL2(1-133)-Ser125 103b (5.0 mg, 0.3 μmol, 1.0 equiv) was dissolved in a 50% aqueous solution of acetic acid (1.29 mL, 0.25 mM) containing 1% AgOAc, then the mixture was vortexed for 2 h at 50° C. in the dark. 50% aqueous solution of acetic acid containing 10% DTT (2.0 ml) was added to the mixture, then the formed precipitate was separated after centrifugation. The precipitate was repeatedly washed with same solution and the combined supernatant (ca. 8 mL) was purified by preparative HPLC using a Shiseido capceil Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2(1-133)-Ser125-SH 107b (3.3 mg, 0.2 μmol, 71% yield). Analytical HPLC and ESI-HRMS were used to confirm the purity and identity of NH₂-IL2(1-133)-Ser125-SH 107b. m/z calculated for C₆₉₅H₁₁₂₅N₁₇₇O₂₀₅S₂ [M+H]⁺: 15304.2738, measured: 15304.3822.

Synthesis of Folded NH₂-IL2(1-133)-Cys125 104a

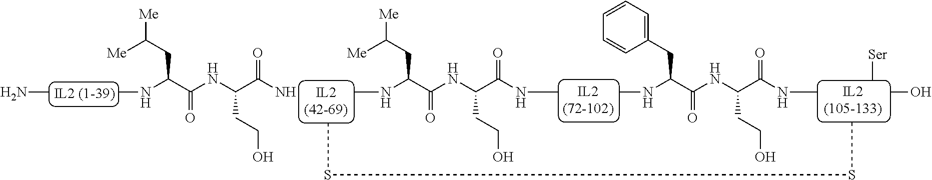

The conditions are disclosed in Asashina, Y. et al.: Chemical Synthesis of O-Glycosylated Human. Interleukin-2 by the Reverse Polarity Protection Strategy. Angew. Chem. Int. Ed. 2015, 54, 8226-8230.

Polypeptide NH₂-IL2(1-133)-SH 107a (6.00 mg, 391 nmol, 1.00 equiv) was dissolved in 6 M Gu•HCl aq. (28.0 mL) containing 0.1 M Tris and 30 mM reduced glutathione, which was adjusted to pH 8.0 by 6 M aq. HCl. The mixture was stored for 1 hat 50° C. A 0.1 M Tris buffer (56.0 mL) containing 1.5 mM oxidized glutathione, which was adjusted to pH 8.0 by 6 M HCl, was added to the mixture was stored for 24 h at rt. The mixture was concentrated in 20-mL spin filters to a final volume of 10 mL, acidified with aqueous TFA and purified by preparative HPLC using a Phenomenex Jupiter C4 column (30×250 mm) with a gradient of 30 to 80% CH₃CN with 0.1% TFA in 30 min, flow rate 10 mL/min. The fractions containing the ligated product were pooled and lyophilized to give pure folded NH₂-IL2(1-133)-Cys125 104a (1.5 mg, 97 nmol, 25% yield). Analytical HPLC and ESI-HRMS were used to confirm the purity and identity of folded NH₂-IL2 (1-133)-Cys125 104a. m/z calculated for C₆₉₅H₁₁₂₃N₁₇₇O₂₀₄S₃ [M+H]⁺: 15318.2349, measured 15318.2749.

Synthesis of Folded NH₂-IL2(1-133)-Ser125 104b

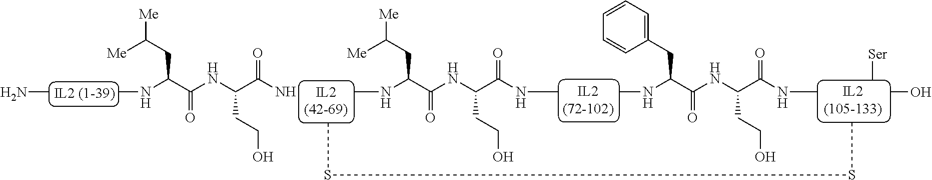

Polypeptide NH₂-IL2(1-133)-Ser125-SH 107b (2.50 mg, 163 nmol, 1.00 equiv) was dissolved in 6 M Gu•HCl aq. (11.0 mL) containing 0.1 M Tris and 30 mM reduced glutathione, which was adjusted to pH 8.0 by 6 M aq, HCl. The mixture was stored for 1 h at 50° C. A 0.1 M Tris buffer (22.0 mL) containing 1.5 mM oxidized glutathione, which was adjusted to pH 8.0 by 6 M HCl, was added to the mixture was stored for 24 h at rt. The mixture was concentrated in 20-mL spin filters to a final volume of 8 mL, acidified with aqueous TFA and purified by preparative HPLC using a Phenomenex Jupiter C4 column (30×250 mm) with a gradient of 30 to 80% CH₃CN with 0.1% TFA in 30 min, flow rate 10 mL/min. The fractions containing the ligated product were pooled and lyophilized to give pure folded NH₂-IL2(1-133)-Ser125 104b (0.8 mg, 52 nmol, 32% yield). Analytical HPLC and ESI-HRMS were used to confirm the purity and identity of folded NH₂-IL2(1-133)-Ser125 104b. m/z calculated for C₆₉₅H₁₁₂₃N₁₇₇O₂₀₅S₃ [M+H]⁺: 15302.2581, measured 15302.2851.

Synthesis of IL-2 Analogues Containing Penicilline (PEN) Residues

1.3.Synthesis of Segments Containing PEN Residues

Synthesis of Opr-IL2(105-133)-PEN S31

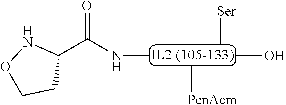

Opr-IL2(105-133)-PEN S31 was synthesized on 2-chlorotrityl polystyrene resin preloaded with Fmoc-Thr(OtBu)-OH and a loading of 0.25 mmol/g. The synthesis was performed on 0.12 mmol scale (0.5 g of resin, 1.0 equiv) by automated Fmoc-SPPS up to Cys105 using the procedure described in the General Methods. Double couplings were used after each coupling and cappings were also added after following residues: Trp, Asn, His, Leu, Ile, Val, Arg, Thr, Asn, Phe, Tyr. Fmoc-PEN(Acm)-OH was used for the coupling of Cys105 and Fmoc-Ser(OtBu)-OH was used to replace Cys125. Isoacyldipeptide Ile129-Ser130 and pseudoproline Ala112-Thr113 were used for optimization of the peptide synthesis. After automated Fmcc-SPPS, BocOpr (68 mg, 0.3 mmol, 2.5 equiv to resin) was coupled to the free amine on-resin using HATU (111 mg, 0.29 mmol, 2.43 equiv to resin) and NMM (68 μL, 0.6 mmol, 5.00 equiv to resin) for 2 h. The resin was washed with DMF and CH₂Cl₂ and the peptide was cleaved from resin following Method A. Purification of crude Opr-IL2(105-133)-PEN S31 was performed by preparative HPIJC using Shiseido Capcell. Pak UG80 C18 column (50×250 mm) preheated to 60° C., with a gradient of 40 to 80% CH3CN with 0.1% TFA in 30 min, flow rate 40 mL/min. The pure product fractions were pooled and lyophilized to obtain Opr-IL2 (105-133)-PEN S31 (30 mg, 8.4 μmol, 7.0% yield for peptide synthesis, resin cleavage and purification steps). Analytical HPLC and MALDI FTMS were used to confirm the purity and exact mass of the product 331. m/z calculated for C₁₆₀H₂₄₅N₃₇O₅₂S [M+H]⁺: 3551.7722; measured 3551.7522.

Synthesis of Opr-IL2(42-69)-PEN-Photoprotected Leu-α-ketoacid S30

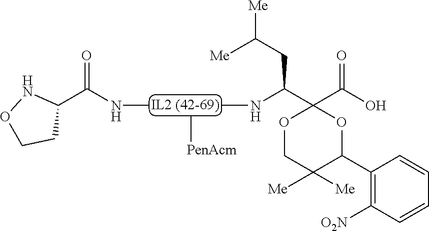

Opr-IL2(42-69)-PEN-Photoprotected Leu-α-ketoacid S30 was synthesized on Rink-Amide Chemmatrix resin (1.0 g, 0.56 mmol/g) preloaded with Fmoc-Photoprotected-Leu-α-ketoacid with a determined loading of 0.15 mmol/g. The synthesis was performed on 0.15 mmol scale (1.0 g of resin, 1.0 equiv) by automated Fmoc-SPPS up to Phe42 using the procedure described in the General Methods. FMoc-PEN(Acm)-OH was used for the coupling of Cys58. Fmoc-Nle-OH was used to replace of Met46. After automated Fmoc-SPPS, BocOpr (33 mg, 0.15 mmol, 2.0 equiv to resin) was coupled to the free amine on-resin using HATU (55 mg, 0.14 mmol, 1.95 equiv to resin) and NMM (33 μL, 0.3 mmol, 4.0 equiv to resin) for 2 h. The resin was washed with DMF and CH₂Cl₂ and the peptide was cleaved from resin following Method B. Purification of crude Opr-IL2(42-69)-PEN-Photoprotected Leu-α-ketoacid S30 was performed by preparative HPLC using Shiseido Capcell Pak UG80 C18 column (50×250 mm) with a gradient of 25 to 60% CH₃CN with 0.1% TFA in 30 min, flow rate 40 mL/min. The pure product fractions were pooled and lyophilized to obtain Opr-IL2(42-69)-PEN-Photoprotected Leu-α-ketoacid S30 (68.0 mg, 17.2 μmol, 24.3% yield for s peptide synthesis, resin cleavage and purification steps). Analytical HPLC was used to confirm the purity of the product S30.

1.4. Assembly to the IL-2 Analogues Containing PEN Residues

1.4.1. Analogue 1

Synthesis of NH₂-IL2(1-69)-PEN-Leu-α-Ketoacid S32

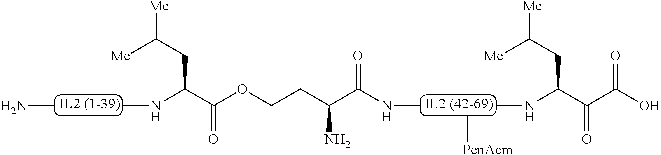

NH₂-IL2(1-39)-Leu-α-ketoacid 90 (33 mg, 7.2 μmol, 1.3 equiv) and Opr-IL2(42-69)-PEN-Photoprotected Leu αα-ketoacid S30 (22.0 mg, 5.60 μmol, 1.00 equiv) were dissolved in 9:1 DMSO:H₂O with 0.1 M oxalic acid (280 μL, 20 mM) and the solution was shaken at 60° C. The progress of the ligation was monitored by analytical HPLC using the same conditions as for NH₂-IL2(1-69)-Leu-α-ketoacid 102. After completion of the ligation (16 h), the reaction mixture was diluted to 4 mL with 1:1 CH₃CN:H₂O with 0.1% TFA and irradiated at a wavelength of 365 nm for 45 min. The reaction mixture was purified by preparative HPLC using a Shiseido Capcell Pak UG80 C18 column (50×250 mm) with a gradient of 30 to 60% CH₃CN with 0.1% TFA in 30 min, flow rate 40 mL/min. The fractions containing the ligated product were pooled and lyophilized to give pure NH₂-IL2(1-69)-PEN-Leu-α-ketoacid S32 (25 mg, 3.0 μmol, 54% yield for ligation and UV deprotection steps). Analytical HPLC and ESI-HRMS were used to confirm the purity and identity of NH₂-IL2(1-69)-PEN-Leu-α-ketoacid S32. m/z measured for NH₂-IL2(1-69)-PEN-Leu-α-ketoacid S32 C₃₇₆H₆₁₉N₉₅O₁₀₈S [M+H]⁺: 8258.64.

Synthesis of NH₂-IL2 (1-133)-PEN(Acm) CysAcm S34

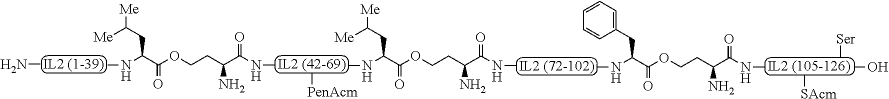

NH₂-IL2(1-69)-PEN-Leu-α-ketoacid S32 (12 mg, 1.4 μmol, 1.3 equiv) and Opr-IL2(72-133)-Ser125 100b (8.0 mg, 1.1 μmol, 1.0 equiv) were dissolved in 9:1 DMSO:H₂O with 0.1 M oxalic is acid (97 μL, 20 mM) and the solution was shaken at 60° C. The progress of the ligation was monitored by analytical HPLC using the same conditions as for NH₂-IL2(1-133)-Acm 103a. After completion of the ligation (16 h), the reaction mixture was diluted to 8 mL with DMSO and purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2(1-133)-PEN(Acm) CysAcm S34 (9.00 mg, 582 nmol, 53.0% yield). Analytical HPLC and ESI-HRMS were used to confirm the purity and identity of NH₂-IL2(1-133)-PEN(Acm) CysAcm S34. m/z calculated for C₇₀₃H₁₁₃₉N₁₇₉O₂₀₇S₂ [M+H]⁺: 15474.3793, measured: 15474.4314.

Synthesis of NH₂-IL2(1-133)-SH-PENCys S37

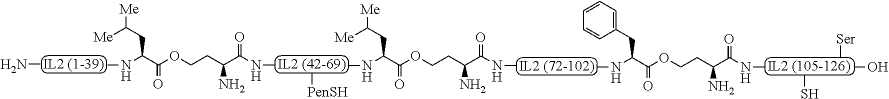

NH₂-IL2(1-133)-PEN(Acm) CysAcm S34 (7.00 mg, 452 nmol, 1.00 equiv) was dissolved in a 50% aqueous solution of acetic acid (1.80 mL, 0.25 mM) containing 1% AgOAc, then the mixture was vortexed for 2 h at 50° C. in the dark. 50% aqueous solution of acetic acid containing 10% DTT (3.0 ml) was added to the mixture, then the formed precipitate was separated after centrifugation. The precipitate was repeatedly washed with same solution and the combined supernatant (ca. 10 mL) was purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2(1-133)-SH-PENCys S37 (5.50 mg, 358 nmol, 79% yield). Analytical HPLC and ESI-HRMS were used to confirm the purity and identity of NH₂-IL2(1-133)-SH-PENCys S37. m/z calculated for C₆₉₇H₁₁₂₉N₁₇₇O₂₀₅S₂ [M+H]⁺: 15332.3051, measured: 15332.3779.

Synthesis of Folded NH₂-IL2(1-133)-PENCys 108

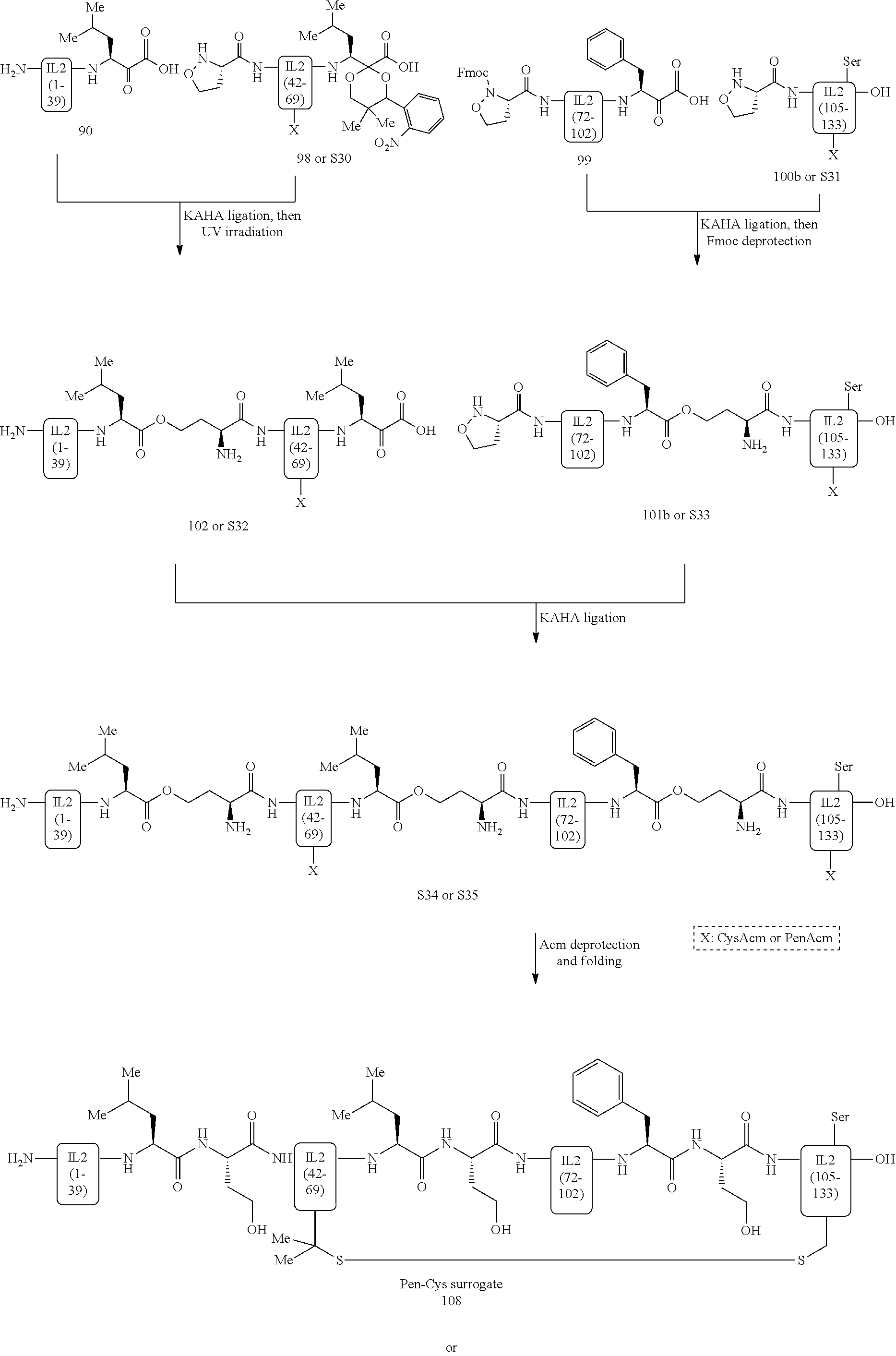

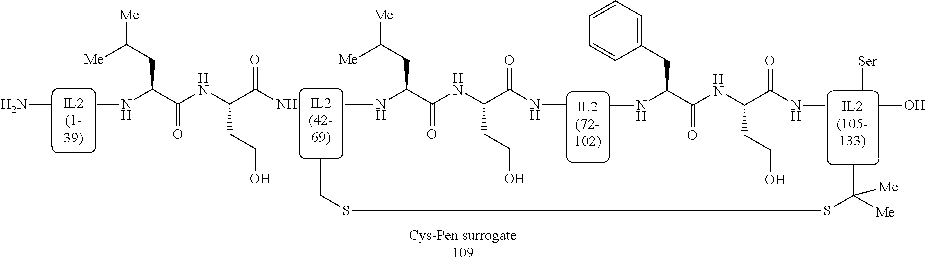

Polypeptide NH₂-IL2(1-133)-SH-PENCys S37 (4.00 mg, 260 nmol, 1.00 equiv) was dissolved in 6 M Gu•HCl aq. (18.0 mL) containing 0.1 M Tris and 30 mM reduced glutathione, which was adjusted to pH 8.0 by 6 M aq. HCl. The mixture was stored for 1 hat 50° C. A 0.1 M Tris buffer (36.0 mL) containing 1.5 mM oxidized glutathione, which was adjusted to pH 8.0 by 6 M HCl, was added to the mixture was stored for 24 h at rt. The mixture was concentrated in 20-mL spin filters to a final volume of 8 mL, acidified with aqueous TFA and purified by preparative HPLC using a Phenomenex Jupiter C4 column (30×250 mm) with a gradient of 30 to 80% CH₃CN with 0.1% TFA in 30 min, flow rate 10 mL/min. The fractions containing the ligated product were pooled and lyophilized to give pure folded NH₂-IL2(1-133)-PENCys 108 (0.5 mg, 65 nmol, 12% yield). Analytical HPLC and ESI-HRMS were used to confirm the purity and identity of folded NH₂-IL2(1-133)-PENCys 108. m/z measured for C₆₉₇H₁₁₂₇N₁₇₇O₂₀₅S₂ [M+H]⁺: 15331.3074.

1.4.2.Analogue 2

Synthesis of Opr-IL2(72-133)-PEN S33

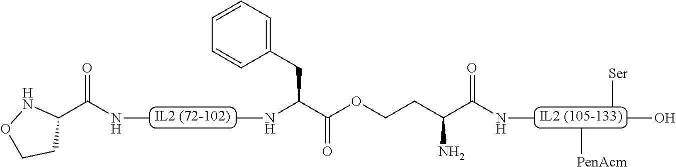

Opr-IL2(102-133)-PEN S31 (14 mg, 3.9 μmol, 1.0 equiv) and FmocOpr-IL2(72-102)-αα-Phe-ketoacid 99 (21 mg, 5.1 μmol, 1.3 equiv) were dissolved in 9:1 DMSO: H₂O with 0.1 M oxalic acid (197 μL, 20 mM) and the solution was shaken at 60° C. The progress of the ligation was monitored by analytical HPLC using using the same conditions as for NH₂-IL2(72-133)-Acm 101a. An aliquot of the ligation mixture (0.1 μL) was taken at various time point, diluted to 12 μL with 1:1 CH₃CN:H₂O and injected on HPLC. After completion of the ligation (16 h), the reaction mixture was diluted to 2 mL with DMSO and 60 μL diethylamine was added dropwise and the solution was shaken for 7 min at rt. The reaction mixture was diluted to 8 mL with DMSO and purified by preparative HPLC using a Shiseido Capcell Pak UG80 C18 column (50×250 mm) preheated to 60° C., with a gradient of 30 to 80% CH₃CN with 1% TFA in 30 min, flow rate 40 mL/min. The fractions containing the ligated product were pooled and lyophilized to give pure Opr-IL2(72-133)-PEN S33 (11 mg, 1.5 μmol, 48% yield for ligation and Fmoc-deprotection steps). Analytical HPLC and ESI-HRMS were used to confirm the purity and identity of Opr-IL2 (72-133)-PEN S33 m/z measured for C₃₂₈H₅₂₀N₈₄O₁₀₁S [M+H]⁺: 7287.84.

Synthesis of NH₂-IL2 (1-133)-CysAcmPEN (Acm) S35

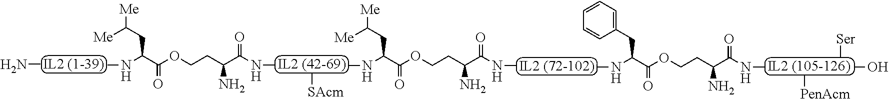

NH₂-IL2(1-69)-Leu-α-ketoacid 102 (12 mg, 1.4 μmol, 1.3 equiv) and Opr-IL2(72-133)-PEN S33 (8.0 mg, 1.1 μmol, 1.0 equiv) were dissolved in 9:1 DMSO:H₂O with 0.1 M oxalic acid (97 μL, 20 mM) and the solution was shaken at 60° C. The progress of the ligation was monitored by analytical HPLC using the same conditions as for IL2 (1-133)-Acm 103a. After completion of the ligation (16 h), the reaction mixture was diluted to 8 mL with DMSO and purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2 (1-133)-CysAcmPEN(Acm) S35 (9.00 mg, 582 nmol, 53.0% yield). Analytical HPLC and ESI-HRMS were used to confirm the purity and identity of NH₂-IL2 (1-1 3)-CysAcmPEN(Acm) S35. m/z calculated for C₇₀₃H₁₁₃₉N₁₇₉O₂₀₇S₂ [M+H]⁺: 15474.3793, measured: 15474.4314.

Synthesis of NH₂-IL2(1-133)-SH-CysPEN S38

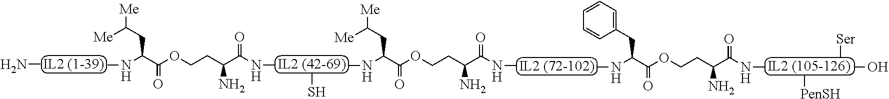

NH₂-IL2(1-133)-CysAcmPEN(Acm) S35 (7.00 mg, 452 nmol, 1.00 equiv) was dissolved in a 50% aqueous solution of acetic acid (1.80 mL, 0.25 mM) containing 1% AgOAc, then the mixture was vertexed for 2 h at 50° C. in the dark. 50% aqueous solution of acetic acid containing 10% DTT (3.0 ml) was added to the mixture, then the formed precipitate was separated after centrifugation. The precipitate was repeatedly washed with same solution and the combined supernatant (ca, 10 mL) was purified by preparative HPLC using a Shiseido Capcell Pak UG80 C18 column (50×250 mm) preheated to 60° C., with a gradient of 30 to 80% CH₃CN with 0.1% TFA in 30 min, flow rate 40 mL/min. The fractions containing the ligated product were pooled and lyophilized to give pure NH₂-IL2(1-133)-SH-CysPEN S38 (5.50 mg, 358 nmol, 79% yield). FSI-HRMS was used to confirm the identity of NH₂-IL2(1-133)-SH-CysPEN 338. m/z calculated for C₆₉₇H₁₁₂₉N₁₇₇O₂₀₅S₂ [M+H]⁺: 15332.3051, measured: 15332.3779.

Synthesis of Folded NH₂-IL2(1-133)-CysPEN 109

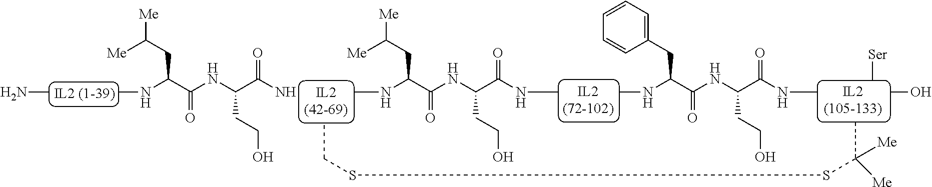

Polypeptide NH₂-IL2(1-133)-SH-CysPEN S38 (4.00 mg, 260 nmol, 1.00 equiv) was dissolved in 6 M Gu•HCl aq. (18.0 mL) containing 0.1 M Tris and 30 mM reduced glutathione, which was adjusted to pH 8.0 by 6 M aq. HCl. The mixture was stored for 1 hat 50° C. A 0.1 M Tris buffer (36.0 mL) containing 1.5 mM oxidized glutathione, which was adjusted to pH 8.0 by 6 M HCl, was added to the mixture was stored for 24 h at rt. The mixture was concentrated in 20-mL spin filters to a final volume of 8 acidified with aqueous TFA and purified by preparative HPLC using a Phenomenex Jupiter C4 column (30×2.50 mm) with a gradient of 30 to 80% CH₃CN with 0.1% TFA in 30 min, flow rate 10 mL/min. The fractions containing the ligated product were pooled and lyophilized to give pure folded NH₂-IL2(1-133)-CysPEN 109 (0.5 mg, 65 nmol, 12% yield). Analytical HPLC and ESI-HRMS were used to confirm the purity and identity of folded NH₂-IL2 (1-133)-CysPEN 109. m/z calculated for C₆₉₇H₁₁₂₇N₁₇₇O₂₀₅S₂ [M+H]⁺: 15331.2889, measured 15331.3074.

Analogue 3

Synthesis of Folded NH₂-IL2(1-133)-Ser125-Methylene Bridge 110

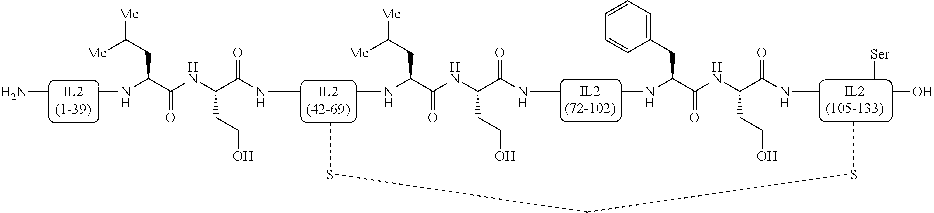

Polypeptide NH₂-IL2(1-133)-OH 104b (0.5 mg, 33 nmol, 1.0 equiv) was dissolved in water (4.5 mL) then added a solution containing TCEP•HCl (47 mg, 0.16 mmol) and K₂CO₃ (47 mg, 0.34 mmol) in 1 ml of water and stored for 5 h. To this mixture, 5% Et₃N in THF (500 μL) and 2% CH₂I₂ (500 μL) were added and stored for 17 h. The reaction mixture was concentrated in 20-mL spin filers to a final volume of 400 μL and purified by analytical HPLC using a Shiseido Capcell park C18 column (10×250 mm) with a gradient of 40 to 95% CH₃CN with 0.1% TFA in 22 min, flow rate 1 mL/min. The fractions containing the ligated product were pooled and lyophilized to give pure folded NH₂-IL2(1-133)-Ser125-methylene bridge 110. ESI-HRMS was used to confirm the identity of folded NH₂-IL2 (1-133)-Ser125-methylene bridge 110. m/z calculated for C₆₉₅H₁₁₂₃N₁₇₇O₂₀₅S₃ [M+H]⁺: 15317.8180, measured 15316.2685.

1. Method for preparing interleukin-2 or an interleukin-2 analogue formed by at least three building blocks comprising the steps of
a. synthesizing the at least three building blocks, whereby the C-terminal amino acid of each building block is linked to an a-keto group and the N-terminal amino acid of each
b. building block is linked to a cyclic hydroxylamine,
c. coupling the at least three building blocks by KAHA ligation resulting in a depsipeptide,
d. rearrangement of the depsipeptide to obtain interleukin-2 or an interleukin-2 analogue.

2. Method according to claim 1, wherein the cyclic hydroxylamine is 5-oxaproline or oxazetidine, preferably 5-oxaproline.

3. Method according to claim 1, wherein interleukin-2 or the interleukin-2 analogue is formed by 3 to 8 building blocks, preferably by 4 building blocks.

4. Method according to claim 1, wherein interleukin-2 or the interleukin-2 analogue is formed by 4 building blocks, and
building block 1 is formed by amino acids 1 to 40 (SEQ. ID. No. 31), building block 2 is formed by amino acids 41 to 70 (SEQ. ID. No. 32), building block 3 is formed by amino acids 71 to 103 ((SEQ. ID. No. 33) and building block 4 is formed by amino acids 104 to 133 (SEQ. ID. No. 34).

5. Method according to claim 1, wherein the C-terminus of the at least three building blocks is selected from the group consisting of leucine, phenylalanine, valine, tyrosine, arginine, glutamine, alanine, norleucine and isoleucine, preferably of leucine, phenylalanine, valine, tyrosine and arginine, and most preferably of leucine, phenylalanine and valine.

6. Method according to claim 1, wherein in step a) at least one, preferably all cysteines at positions 58, 105 and 125 of the interleukin-2 sequence (SEQ. ID. NO. 1) are replaced by cysteine S-acetamidomethyl (CysAcm).

7. Method according to claim 1, wherein the rearrangement of the depsipeptide is carried out in a basic buffer at a pH ranging from 8 to 10.

8. Method for preparing an interleukin-2 analogue according to claim 1, wherein one of the cysteines at positions 58 and 105 of the interleukin-2 sequence (SEQ. ID. NO. 1) forming a disulfide bond is replaced by a non-reducible surrogate.

9. Method according to claim 8, wherein the analogue is a variant of interleukin-2 sequence (SEQ. ID. NO. 1) comprising a substitute at the following amino acid positions Cys58 or Cys105 and the substitute is preferably penicillamine (PEN).

10. Method according to claim 1, wherein the analogue is a variant of interleukin-2 sequence (SEQ. ID. NO. 1) comprising substitutes at amino acid position Cys125 and the substitute is preferably serine (Ser).

11. Method according to claim 1, wherein the analogue is a variant of interleukin-2 sequence (SEQ. ID. NO. 1) comprising substitutes at one or more of the following amino acid position Met23, Met39 and Met46 and the substitute is preferably norleucine (Nle).

12. Method according to claim 1, wherein the analogue is a variant of interleukin-2 sequence (SEQ. ID. NO. 1) comprising substitutes at one or more of the following amino acid positions Tyr41, Asn71 and Met104 and the substitute is preferably homoserine (Hse).

13. Method according to claim 1, wherein the disulfide bond formed by the amino acids at positions 58 and 105 is replaced by a methylene thioacetal bridge.

14. Interleukin-2 analogues selected from the group consisting of SEQ. ID. No. 2, SEQ. ID. No. 3, SEQ. ID. No. 4, SEQ. ID. No. 5, SEQ. ID. No. 6, SEQ. ID. No. 7, SEQ. ID. No. 8, SEQ. ID. No. 9, SEQ. ID. No. 10, SEQ. ID. No. 11, SEQ. ID. No. 12, SEQ. ID. No. 13, SEQ. ID. No. 14, SEQ. ID. No. 15, SEQ. ID. No. 16, SEQ. ID. No. 17, SEQ. ID. No. 18, SEQ. ID. No. 19, SEQ. ID. No. 20, SEQ. ID. No. 21, SEQ. ID. No. 22, SEQ. ID. No. 23, SEQ. ID. No. 24, SEQ. ID. No. 25, SEQ. ID. No. 26, SEQ. ID. No. 27, SEQ. ID. No. 28, and SEQ. ID. No. 29.

15. Building blocks for preparing interleukin-2 analogues selected from the group consisting of SEQ. ID. No. 30, SEQ. ID. No. 31, and SEQ. ID. No. 32, SEQ. ID. No. 33, SEQ. ID. No. 34, SEQ. ID. No. 35, SEQ. ID. No. 36, and SEQ. ID. No. 37.


SEQUENCE LISTING (37 sequences)

SEQ ID NO: 1 - protein, 133 aa, Homo sapiens [sequence omitted]

SEQ ID NO: 2 - protein, 133 aa, Artificial Sequence
  modified Interleukin-2
  MOD_RES 58..58: Penicillamine (PEN)
APTSSSTKKTQLQLEHLLLDLQMILNGINNYKNPKLTRMLTFKFYMPKKATELKHLQXLEEELKPLEEVLNLAQSKNFHLRPRDLISNINVIVLELKGSETTFMCEYADETATIVEFLNRWITFCQSIISTLT

SEQ ID NO: 3 - protein, 133 aa, Artificial Sequence
  modified Interleukin-2
  MOD_RES 105..105: Penicillamine (PEN)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMXEYADETATIVEFLNRWITFCQSIISTLT

SEQ ID NO: 4 - protein, 133 aa, Artificial Sequence
  Modified Interleukin-2
  MOD_RES 58..58: Penicillamine (PEN)
APTSSSTKKTQLQLEHLLLDLQMILNGINNYKNPKLTRMLTFKFYMPKKATELKHLQXLEEELKPLEEVLNLAQSKNFHLRPRDLISNINVIVLELKGSETTFMCEYADETATIVEFLNRWITFSQSIISTLT

SEQ ID NO: 5 - protein, 133 aa, Artificial Sequence
  Modified Interleukin-2
  MOD_RES 105..105: Penicillamine (PEN)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMXEYADETATIVEFLNRWITFSQSIISTLT

SEQ ID NO: 6 - protein, 133 aa, Artificial Sequence
  Modified Interleukin-2
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 7 - protein, 133 aa, Artificial Sequence
  Modified Interleukin-2
  MOD_RES 23..23: Norleucine (Nle)
APTSSSTKKTQLQLEHLLLDLQXILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 8 - protein, 133 aa, Artificial Sequence
  Modified Interleukin-2
  MOD_RES 39..39: Norleucine (Nle)
APTSSSTKKTQLQLEHLLLDLQMILNGINNYKNPKLTRXLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 9 - protein, 133 aa, Artificial Sequence
  Modified Interleukin-2
  MOD_RES 46..46: Norleucine (Nle)
APTSSSTKKTQLQLEHLLLDLQMILNGINNYKNPKLTRMLTFKFYXPKKATELKHLQCLEEELKPLEEVLNLAQSKNFHLRPRDLISNINVIVLELKGSETTFMCEYADETATIVEFLNRWITFCQSIISTLT

SEQ ID NO: 10 - protein, 133 aa, Artificial Sequence
  Modified Interleukin-2
  MOD_RES 23..23: Norleucine (Nle)
  MOD_RES 39..39: Norleucine (Nle)
APTSSSTKKTQLQLEHLLLDLQXILNGINNYKNPKLTRXLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 11 - protein, 133 aa, Artificial Sequence
  Modified Interleukin-2
  MOD_RES 23..23: Norleucine (Nle)
  MOD_RES 46..46: Norleucine (Nle)
APTSSSTKKTQLQLEHLLLDLQXILNGINNYKNPKLTRMLTFKFYXPKKATELKHLQCLEEELKPLEEVLNLAQSKNFHLRPRDLISNINVIVLELKGSETTFMCEYADETATIVEFLNRWITFCQSIISTLT

SEQ ID NO: 12 - protein, 133 aa, Artificial Sequence
  Modified Interleukin-2
  MOD_RES 39..39: Norleucine (Nle)
  MOD_RES 46..46: Norleucine (Nle)
APTSSSTKKTQLQLEHLLLDLQMILNGINNYKNPKLTRXLTFKFYXPKKATELKHLQCLEEELKPLEEVLNLAQSKNFHLRPRDLISNINVIVLELKGSETTFMCEYADETATIVEFLNRWITFCQSIISTLT

SEQ ID NO: 13 - protein, 133 aa, Artificial Sequence
  Modified Interleukin-2
  MOD_RES 23..23: Norleucine (Nle)
  MOD_RES 39..39: Norleucine (Nle)
  MOD_RES 46..46: Norleucine (Nle)
APTSSSTKKTQLQLEHLLLDLQXILNGINNYKNPKLTRXLTFKFYXPKKATELKHLQCLEEELKPLEEVLNLAQSKNFHLRPRDLISNINVIVLELKGSETTFMCEYADETATIVEFLNRWITFCQSIISTLT

SEQ ID NO: 14 - protein, 133 aa, Artificial Sequence
  Modified Interleukin-2
  MOD_RES 41..41: Homoserine (Hse)
APTSSSTKKTQLQLEHLLLDLQMILNGINNYKNPKLTRMLXFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 15 - protein, 133 aa, Artificial Sequence
  Modified Interleukin-2
  MOD_RES 71..71: Homoserine (Hse)
APTSSSTKKTQLQLEHLLLDLQMILNGINNYKNPKLTRMLTFKFYMPKKATELKHLQCLEEELKPLEEVLXLAQSKNFHLRPRDLISNINVIVLELKGSETTFMCEYADETATIVEFLNRWITFCQSIISTLT

SEQ ID NO: 16 - protein, 133 aa, Artificial Sequence
  Modified Interleukin-2
  MOD_RES 104..104: Homoserine (Hse)
APTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFXCEYADETATIVEFLNRWITFCQSIISTLT

SEQ ID NO: 17 - protein, 133 aa, Artificial Sequence
  Modified Interleukin-2
  MOD_RES 41..41: Homoserine (Hse)
  MOD_RES 71..71: Homoserine (Hse)
  MOD_RES 104..104: Homoserine (Hse)
APTSSSTKKTQLQLEHLLLDLQMILNGINNYKNPKLTRMLXFKFYMPKKATELKHLQCLEEELKPLEEVLXLAQSKNFHLRPRDLISNINVIVLELKGSETTFXCEYADETATIVEFLNRWITFCQSIISTLT

SEQ ID NO: 18 - protein, 133 aa, Artificial Sequence
  Modified Interleukin-2
  MOD_RES 23..23: Norleucine (Nle)
  MOD_RES 39..39: Norleucine (Nle)
  MOD_RES 46..46: Norleucine (Nle)
  MOD_RES 58..58: Penicillamine (PEN)
APTSSSTKKTQLQLEHLLLDLQXILNGINNYKNPKLTRXLTFKFYXPKKATELKHLQXLEEELKPLEEVLNLAQSKNFHLRPRDLISNINVIVLELKGSETTFMCEYADETATIVEFLNRWITFCQSIISTLT

SEQ ID NO: 19 - protein, 133 aa, Artificial Sequence
  Modified Interleukin-2
  MOD_RES 23..23: Norleucine (Nle)
  MOD_RES 39..39: Norleucine (Nle)
  MOD_RES 46..46: Norleucine (Nle)
  MOD_RES 105..105: Penicillamine (PEN)
APTSSSTKKTQLQLEHLLLDLQXILNGINNYKNPKLTRXLTFKFYXPKKATELKHLQCLEEELKPLEEVLNLAQSKNFHLRPRDLISNINVIVLELKGSETTFMXEYADETATIVEFLNRWITFCQSIISTLT

SEQ ID NO: 20 - protein, 133 aa, Artificial Sequence
  Modified Interleukin-2
  MOD_RES 23..23: Norleucine (Nle)
  MOD_RES 39..39: Norleucine (Nle)
  MOD_RES 46..46: Norleucine (Nle)
  MOD_RES 58..58: Penicillamine (PEN)
APTSSSTKKTQLQLEHLLLDLQXILNGINNYKNPKLTRXLTFKFYXPKKATELKHLQXLEEELKPLEEVLNLAQSKNFHLRPRDLISNINVIVLELKGSETTFMCEYADETATIVEFLNRWITFSQSIISTLT

SEQ ID NO: 21 - protein, 133 aa, Artificial Sequence
  Modified Interleukin-2
  MOD_RES 23..23: Norleucine (Nle)
  MOD_RES 39: Norleucine (Nle)
  MOD_RES 46..46: Norleucine (Nle)
  MOD_RES 105..105: Penicillamine (PEN)
APTSSSTKKTQLQLEHLLLDLQXILNGINNYKNPKLTRXLTFKFYXPKKATELKHLQCLEEELKPLEEVLNLAQSKNFHLRPRDLISNINVIVLELKGSETTFMXEYADETATIVEFLNRWITFSQSIISTLT

SEQ ID NO: 22 - protein, 133 aa, Artificial Sequence
  Modified Interleukin-2
  MOD_RES 41..41: Homoserine (Hse)
  MOD_RES 58..58: Penicillamine (PEN)
  MOD_RES 71..71: Homoserine (Hse)
  MOD_RES 104..104: Homoserine
APTSSSTKKTQLQLEHLLLDLQMILNGINNYKNPKLTRMLXFKFYMPKKATELKHLQXLEEELKPLEEVLXLAQSKNFHLRPRDLISNINVIVLELKGSETTFXCEYADETATIVEFLNRWITFCQSIISTLT

SEQ ID NO: 23 - protein, 133 aa, Artificial Sequence
  Modified Interleukin-2
  MOD_RES 41..41: Homoserine (Hse)
  MOD_RES 71..71: Homoserine (Hse)
  MOD_RES 104..104: Homoserine (Hse)
  MOD_RES 105..105: Penicillamine (PEN)
APTSSSTKKTQLQLEHLLLDLQMILNGINNYKNPKLTRMLXFKFYMPKKATELKHLQCLEEELKPLEEVLXLAQSKNFHLRPRDLISNINVIVLELKGSETTFXXEYADETATIVEFLNRWITFCQSIISTLT

SEQ ID NO: 24 - protein, 133 aa, Artificial Sequence
  Modified Interleukin-2
  MOD_RES 41..41: Homoserine (Hse)
  MOD_RES 58..58: Penicillamine (PEN)
  MOD_RES 71..71: Homoserine (Hse)
  MOD_RES 104..104: Homoserine (Hse)
APTSSSTKKTQLQLEHLLLDLQMILNGINNYKNPKLTRMLXFKFYMPKKATELKHLQXLEEELKPLEEVLXLAQSKNFHLRPRDLISNINVIVLELKGSETTFXCEYADETATIVEFLNRWITFSQSIISTLT

SEQ ID NO: 25 - protein, 133 aa, Artificial Sequence
  Modified Interleukin-2
  MOD_RES 41..41: Homoserine (Hse)
  MOD_RES 71..71: Homoserine (Hse)
  MOD_RES 104..104: Homoserine (Hse)
  MOD_RES 105..105: Penicillamine (PEN)
APTSSSTKKTQLQLEHLLLDLQMILNGINNYKNPKLTRMLXFKFYMPKKATELKHLQCLEEELKPLEEVLXLAQSKNFHLRPRDLISNINVIVLELKGSETTFXXEYADETATIVEFLNRWITFSQSIISTLT

SEQ ID NO: 26 - protein, 133 aa, Artificial Sequence
  Modified Interleukin-2
  MOD_RES 23..23: Norleucine (Nle)
  MOD_RES 39..39: Norleucine (Nle)
  MOD_RES 41..41: Homoserine (Hse)
  MOD_RES 46..46: Norleucine (Nle)
  MOD_RES 58..58: Penicillamine (PEN)
  MOD_RES 71..71: Homoserine (Hse)
  MOD_RES 104..104: Homoserine (Hse)
APTSSSTKKTQLQLEHLLLDLQXILNGINNYKNPKLTRXLXFKFYXPKKATELKHLQXLEEELKPLEEVLXLAQSKNFHLRPRDLISNINVIVLELKGSETTFXCEYADETATIVEFLNRWITFCQSIISTLT

SEQ ID NO: 27 - protein, 133 aa, Artificial Sequence
  Modified Interleukin-2
  MOD_RES 23..23: Norleucine (Nle)
  MOD_RES 39..39: Norleucine (Nle)
  MOD_RES 41..41: Homoserine (Hse)
  MOD_RES 46..46: Norleucine (Nle)
  MOD_RES 71..71: Homoserine (Hse)
  MOD_RES 104..104: Homoserine (Hse)
  MOD_RES 105..105: Penicillamine (PEN)
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXXEYADETATIVEFLNRWITFCQSIISTLT

SEQ ID NO: 28 - protein, 133 aa, Artificial Sequence
  Modified Interleukin-2
  MOD_RES 23..23: Norleucine (Nle)
  MOD_RES 39..39: Norleucine (Nle)
  MOD_RES 41..41: Homoserine (Hse)
  MOD_RES 46..46: Norleucine (Nle)
  MOD_RES 58..58: Penicillamine (PEN)
  MOD_RES 71..71: Homoserine (Hse)
  MOD_RES 104..104: Homoserine (Hse)
APTSSSTKKTQLQLEHLLLDLQXILNGINNYKNPKLTRXLXFKFYXPKKATELKHLQXLEEELKPLEEVLXLAQSKNFHLRPRDLISNINVIVLELKGSETTFXCEYADETATIVEFLNRWITFSQSIISTLT

SEQ ID NO: 29 - protein, 133 aa, Artificial Sequence
  Modified Interleukin-2
  MOD_RES 23..23: Norleucine (Nle)
  MOD_RES 39..39: Norleucine (Nle)
  MOD_RES 41..41: Homoserine (Hse)
  MOD_RES 46..46: NOrleucine (Nle)
  MOD_RES 71..71: Homoserine (Hse)
  MOD_RES 104..104: Homoserine (Hse)
  MOD_RES 105..105: Penicillamine (PEN)
APTSSSTKKTQLQLEHLLLDLQXILNGINNYKNPKLTRXLXFKFYXPKKATELKHLQCLEEELKPLEEVLXLAQSKNFHLRPRDLISNINVIVLELKGSETTFXXEYADETATIVEFLNRWITFSQSIISTLT

SEQ ID NO: 30 - protein, 30 aa, Artificial Sequence
  MOD_RES 1..1: Homoserine (Hse)
  Modified building block 4
XCEYADETATIVEFLNRWITFSQSIISTLT

SEQ ID NO: 31 - protein, 40 aa, Artificial Sequence
  building block 1
  MOD_RES 23..23: Norleucine (Nle)
  MOD_RES 39..39: Norleucine (Nle)
APTSSSTKKTQLQLEHLLLDLQXILNGINNYKNPKLTRXL

SEQ ID NO: 32 - protein, 30 aa, Artificial Sequence
  MOD_RES 1..1: Homoserine (Hse)
  building block 2
  MOD_RES 6..6: Norleucine (Nle)
XFKFYXPKKATELKHLQCLEEELKPLEEVL

SEQ ID NO: 33 - protein, 33 aa, Artificial Sequence
  MOD_RES 1..1: Homoserine (Hse)
  building block 3
XLAQSKNFHLRPRDLISNINVIVLELKGSETTF

SEQ ID NO: 34 - protein, 30 aa, Artificial Sequence
  MOD_RES 1..1: Homoserine (Hse)
  building block 4
XCEYADETATIVEFLNRWITFCQSIISTLT

SEQ ID NO: 35 - protein, 30 aa, Artificial Sequence
  MOD_RES 1..1: Homoserine (Hse)
  Modified Building Block 2
  MOD_RES 6..6: Norleucine (Nle)
  MOD_RES 18..18: Penicillamine (PEN)
XFKFYXPKKATELKHLQXLEEELKPLEEVL

SEQ ID NO: 36 - protein, 30 aa, Artificial Sequence
  MOD_RES 1..1: Homoserine (Hse)
  Modified Building Block 4
  MOD_RES 2..2: Penicillamine (PEN)
XXEYADETATIVEFLNRWITFCQSIISTLT

SEQ ID NO: 37 - protein, 30 aa, Artificial Sequence
  MOD_RES 1..1: Homoserine (Hse)
  Modified Building Block 4
  MOD_RES 2..2: Penicillamine (PEN)
XXEYADETATIVEFLNRWITFSQSIISTLT